(19) United States
(12) Patent Application Publication
(10) Pub. No.: US 2024/0254566 A1
(43) Pub. Date: Aug. 1, 2024

(54) ANALYSIS METHOD, KIT, AND DETECTION DEVICE

(71) Applicant: KABUSHIKI KAISHA TOSHIBA, Tokyo (JP)

(72) Inventors: Takuya MIYAGAWA, Yokohama (JP); Yoshitake SANO, Adachi (JP); Tomomi ANDO, Ota (JP); Mitsuko ISHIHARA, Setagaya (JP); Miho SAKO, Kawasaki (JP); Masaaki TAKADA, Kashiwa (JP); Hisashi YAMADA, Kawasaki (JP)

(73) Assignee: KABUSHIKI KAISHA TOSHIBA, Tokyo (JP)

(21) Appl. No.: 18/591,359

(22) Filed: Feb. 29, 2024

Related U.S. Application Data
(63) Continuation of application No. PCT/JP2023/007849, filed on Mar. 2, 2023.

(30) Foreign Application Priority Data
Aug. 31, 2022 (JP) 2022-138226

(51) Int. Cl. C12Q 1/6886; C12Q 1/6851

(52) U.S. Cl. CPC C12Q 1/6886; C12Q 1/6851; C12Q 2600/166; C12Q 2600/178

(57) ABSTRACT

An analysis method may determine the presence of affection of at least any one of breast cancer, pancreatic cancer, lung cancer, gastric cancer, and colorectal cancer. The analysis method may include quantifying at least any one of hsa-miR-205-5p, hsa-miR-30e-5p, hsa-miR-106b-5p, hsa-miR-3613-5p, hsa-miR-483-5p, hsa-miR-574-3p, hsa-miR-125b-5p, hsa-miR-223-5p, hsa-miR-3613-3p, hsa-miR-941, hsa-miR-324-3p, hsa-miR-193a-5p, hsa-miR-4433a-3p, hsa-miR-29c-3p, hsa-miR-190a-5p, hsa-miR-885-5p, hsa-miR-194-5p, hsa-miR-29a-3p, hsa-miR-142-5p, hsa-miR-142-3p, hsa-miR-122-5p, hsa-miR-34a-5p, and hsa-miR-375-3p in a sample derived from an object.

### CROSS-REFERENCE TO RELATED APPLICATIONS

This application is a Continuation application of PCT Application No. PCT/JP2023/007849, filed Mar. 2, 2023 and is based upon and claiming the benefit of priority from Japanese Patent Application No. 2022-138226, filed Aug. 31, 2022, the entire contents of all of which are incorporated herein by reference.

### REFERENCE TO A SEQUENCE LISTING

In accordance with 37 CFR $1.831, the present specification makes reference to a Sequence Listing submitted electronically as a .xml file named “552167US_Verified_ST26. xml”. The .xml file was generated on Feb. 26, 2024 and is 187, 545 bytes in size. The entire contents of the Sequence Listing are hereby incorporated by reference.

### FIELD

Embodiments described herein relate generally to an analysis method, a kit, and a detection device.

### BACKGROUND

In recent years, a relationship between microRNA (miRNA) and a disease has attracted attention. It has been reported that miRNAs have a function of regulating gene expression, and the type and expression level thereof are changed from an early stage in various diseases. That is, in a patient having a certain disease, the amount of a specific miRNA is increased or decreased as compared with that in a healthy person. Therefore, examining the amount of the miRNA in a sample collected from a subject is a means of knowing whether or not a patient suffers from the disease.

### BRIEF DESCRIPTION OF THE DRAWINGS

FIG. 1 is a flowchart showing an example of an analysis method of a first embodiment, in which (a) is an example of an analysis method including a quantification step of any one of 23 target miRNAs, (b) is an example of an analysis method including a determination step of determining the presence of affection of at least any one of breast cancer, pancreatic cancer, lung cancer, gastric cancer, and colorectal cancer in an object, (c) is an example of an analysis method including a determination step of determining prognosis or the presence of recurrence, and (d) is an example of an analysis method including a selection step of selecting the type of therapy or the type of drug to be applied to an object.

FIG. 2 is a flowchart showing an example of an analysis method of a second embodiment, in which (a) is an example of an analysis method including a quantification step of quantifying a combination of a plurality of specific types of target miRNAs among 23 target miRNAs, (b) is an example of an analysis method including a determination step of determining the presence of affection of at least any one of breast cancer, pancreatic cancer, lung cancer, gastric cancer, and colorectal cancer in an object, (c) is an example of an analysis method including a determination step of determining prognosis or the presence of recurrence, and (d) is an example of an analysis method including a selection step of selecting the type of therapy or the type of drug to be applied to an object.

FIG. 3A is a box plot showing experimental results of Example 2, showing quantitative values of hsa-miR-205-5p for each type of specimen.

FIG. 3B is a box plot showing experimental results of Example 2, showing quantitative values of hsa-miR-30e-5p for each type of specimen.

FIG. 3C is a box plot showing experimental results of Example 2, showing quantitative values of hsa-miR-106b-5p for each type of specimen.

FIG. 3D is a box plot showing experimental results of Example 2, showing quantitative values of hsa-miR-3613-5p for each type of specimen.

FIG. 3E is a box plot showing experimental results of Example 2, showing quantitative values of hsa-miR-483-5p for each type of specimen.

FIG. 3F is a box plot showing experimental results of Example 2, showing quantitative values of hsa-miR-574-3p for each type of specimen.

FIG. 3G is a box plot showing experimental results of Example 2, showing quantitative values of hsa-miR-125b-5p for each type of specimen.

FIG. 3H is a box plot showing experimental results of Example 2, showing quantitative values of hsa-miR-223-5p for each type of specimen.

FIG. 3I is a box plot showing experimental results of Example 2, showing quantitative values of hsa-miR-3613-3p for each type of specimen.

FIG. 3J is a box plot showing experimental results of Example 2, showing quantitative values of hsa-miR-941 for each type of specimen.

FIG. 3K is a box plot showing experimental results of Example 2, showing quantitative values of hsa-miR-324-3p for each type of specimen.

FIG. 3L is a box plot showing experimental results of Lis Example 2, showing quantitative values of hsa-miR-193a-5p for each type of specimen.

FIG. 3M is a box plot showing experimental results of Example 2, showing quantitative values of hsa-miR-4433a-3p for each type of specimen.

FIG. 3N is a box plot showing experimental results of Example 2, showing quantitative values of hsa-miR-29c-3p for each type of specimen.

FIG. 3O is a box plot showing experimental results of Example 2, showing quantitative values of hsa-miR-190a-5p for each type of specimen.

FIG. 3P is a box plot showing experimental results of Example 2, showing quantitative values of hsa-miR-885-5p for each type of specimen.

FIG. 3Q is a box plot showing experimental results of Example 2, showing quantitative values of hsa-miR-194-5p for each type of specimen.

FIG. 3R is a box plot showing experimental results of Example 2, showing quantitative values of hsa-miR-29a-3p for each type of specimen.

FIG. 3S is a box plot showing experimental results of Example 2, showing quantitative values of hsa-miR-142-5p for each type of specimen.

FIG. 3T is a box plot showing experimental results of Example 2, showing quantitative values of hsa-miR-142-3p for each type of specimen.

FIG. 3U is a box plot showing experimental results of Example 2, showing quantitative values of hsa-miR-122-5p for each type of specimen.

FIG. 3V is a box plot showing experimental results of Example 2, showing quantitative values of hsa-miR-34a-5p for each type of specimen.

FIG. 3W is a box plot showing experimental results of Example 2, showing quantitative values of hsa-miR-375-3p for each type of specimen.

### DETAILED DESCRIPTION

In general, according to one embodiment, an analysis method for determining the presence of affection of at least any one of breast cancer, pancreatic cancer, lung cancer, gastric cancer, and colorectal cancer, including quantifying at least any one of hsa-miR-205-5p, hsa-miR-30e-5p, hsa-miR-106b-5p, hsa-miR-3613-5p, hsa-miR-483-5p, hsa-miR-574-3p, hsa-miR-125b-5p, hsa-miR-223-5p, hsa-miR-3613-3p, hsa-miR-941, hsa-miR-324-3p, hsa-miR-193a-5p, hsa-miR-4433a-3p, hsa-miR-29c-3p, hsa-miR-190a-5p, hsa-miR-885-5p, hsa-miR-194-5p, hsa-miR-29a-3p, hsa-miR-142-5p, hsa-miR-142-3p, hsa-miR-122-5p, hsa-miR-34a-5p, and hsa-miR-375-3p in a sample derived from an object.

Hereinafter, an analysis method, a kit, and a detection device according to embodiments will be described with reference to the drawings.

### First Embodiment

### (Analysis Method)

An analysis method according to a first embodiment is an analysis method for determining the presence of affection of at least any one of breast cancer, pancreatic cancer, lung cancer, gastric cancer, and colorectal cancer, including quantifying one type of hsa-miR-205-5p, hsa-miR-30e-5p, hsa-miR-106b-5p, hsa-miR-3613-5p, hsa-miR-483-5p, hsa-miR-574-3p, hsa-miR-125b-5p, hsa-miR-223-5p, hsa-miR-3613-3p, hsa-miR-941, hsa-miR-324-3p, hsa-miR-193a-5p, hsa-miR-4433a-3p, hsa-miR-29c-3p, hsa-miR-190a-5p, hsa-miR-885-5p, hsa-miR-194-5p, hsa-miR-29a-3p, hsa-miR-142-5p, hsa-miR-142-3p, hsa-miR-122-5p, hsa-miR-34a-5p, and hsa-miR-375-3p in a sample derived from an object (quantification step (S12)).

The total of 23 miRNAs described above are also referred to as “target miRNA group” in the following description. The individual miRNA constituting the target miRNA group is also referred to as “target miRNA”.

For example, each target miRNA is represented by a base sequence shown in Table 1 below. In the present specification, notation “T” on a sequence listing corresponding to each sequence number means “U”. The target miRNA to be quantified may be one type of the target miRNA group.

TABLE 1

| Sequence | | |
|---|---|---|
| number | Name of miRNA | Base sequence |
| 1 | hsa-miR-3613-5p | UGUUGUACUUUUUUUUUUGUUC |
| 2 | hsa-miR-574-3p | CACGCUCAUGCACACACCCACA |
| 3 | hsa-miR-125b-5p | UCCCUGAGACCCUAACUUGUGA |
| 4 | hsa-miR-223-5p | CGUGUAUUUGACAAGCUGAGUU |
| 5 | hsa-miR-3613-3p | ACAAAAAAAAAAGCCCAACCCUUC |
| 6 | hsa-miR-205-5p | UCCUUCAUUCCACCGGAGUCUG |
| 7 | hsa-miR-941 | CACCCGGCUGUGUGCACAUGUGC |
| 8 | hsa-miR-324-3p | CCCACUGCCCCAGGUGCUGCUGG |
| 9 | hsa-miR-30e-5p | UGUAAACAUCCUUGACUGGAAG |
| 10 | hsa-miR-193a-5p | UGGGUCUUUGCGGGCGAGAUGA |
| 11 | hsa-miR-106b-5p | UAAAGUGCUGACAGUGCAGAU |
| 12 | hsa-miR-4433a-3p | ACAGGAGUGGGGGUGGGACAU |
| 13 | hsa-miR-29c-3p | UAGCACCAUUUGAAAUCGGUUA |
| 14 | hsa-miR-190a-5p | UGAUAUGUUUGAUAUAUUAGGU |
| 15 | hsa-miR-885-5p | UCCAUUACACUACCCUGCCUCU |
| 16 | hsa-miR-194-5p | UGUAACAGCAACUCCAUGUGGA |
| 17 | hsa-miR-29a-3p | UAGCACCAUCUGAAAUCGGUUA |
| 18 | hsa-miR-142-5p | CAUAAAGUAGAAAGCACUACU |
| 19 | hsa-miR-483-5p | AAGACGGGAGGAAAGAAGGGAG |
| 20 | hsa-miR-142-3p | UGUAGUGUUUCCUACUUUAUGGA |
| 21 | hsa-miR-122-5p | UGGAGUGUGACAAUGGUGUUUG |
| 22 | hsa-miR-34a-5p | UGGCAGUGUCUUAGCUGGUUGU |
| 23 | hsa-miR-375-3p | UUUGUUCGUUCGGCUCGCGUGA |

The object is an animal objected to analysis in the present method, i.e., an animal providing the sample. The object may be an animal having any disease or a healthy animal. For example, the object may be an animal possibly suffering from cancer, an animal having suffered from cancer in the past, or the like. In particular, the object may be an animal possibly suffering from at least any one of breast cancer, pancreatic cancer, lung cancer, gastric cancer, and colorectal cancer, an animal having suffered from at least any one of breast cancer, pancreatic cancer, lung cancer, gastric cancer, and colorectal cancer in the past, or the like. The object is preferably a human.

Alternatively, the object may be another animal. The another animal is, for example, a mammal, and includes, for example, a primate such as a monkey, a rodent such as a mouse, a rat, or a guinea pig, a companion animal such as a dog, a cat, or a rabbit, a livestock animal such as a horse, a cow, or a pig, or an animal belonging to a display animal or the like. In the case of an animal other than human, the target miRNA is a miRNA corresponding to the miRNA present in the animal.

The sample derived from an object includes a sample collected from an object or a sample obtained by appropriately treating the sample. The sample is preferably serum or plasma. The sample may be another body fluid, for example, blood, leukocyte interstitial fluid, urine, feces, sweat, saliva, intraoral mucosa, intranasal mucosa, nasal discharge, pharyngeal mucosa, sputum, digestive fluid, gastric fluid, lymphatic fluid, spinal fluid, lacrimal fluid, breast milk, amniotic fluid, semen, vaginal fluid, or the like. Alternatively, the sample may be a tissue, a cell, or the like, for example, a tissue or a cell collected from an object and cultured, or a supernatant thereof.

In the present specification, various “cancers” include those at any stage, and include, for example, a state of remaining in an organ of an origin, a state of further extending to a surrounding tissue, a state of further metastasizing to a lymph node, a state of having metastasis to a further distant organ, and the like. In addition, in the present specification, breast cancer refers to a malignant tumor (neoplasm) formed in mammary gland tissue. In addition, the various cancers in the present specification include, for example, an epithelial tumor, a non-epithelial tumor, or a tumor including both epithelial and non-epithelial tumors.

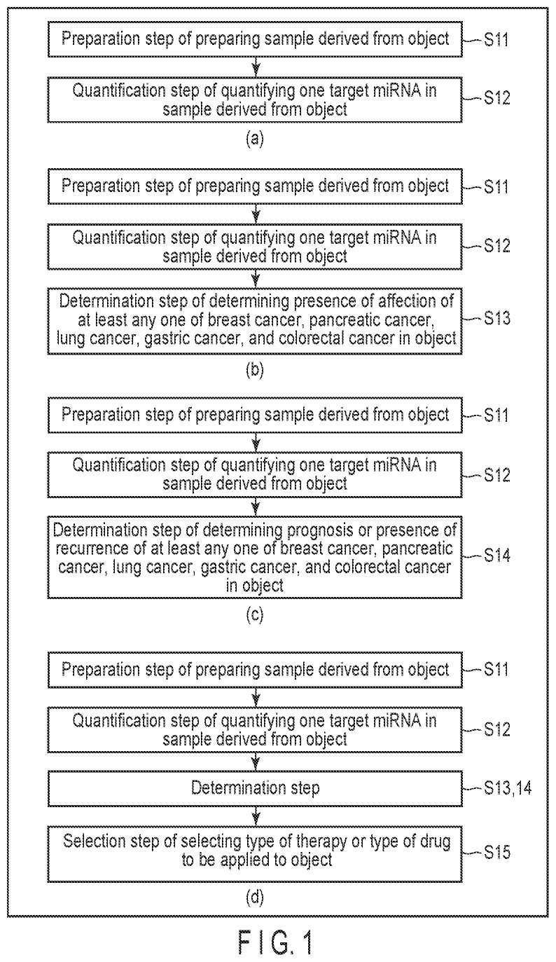

Hereinafter, an example of the procedure of the method of the first embodiment will be described with reference to (a), (b), and (c) of FIG. 1.

As shown in (a) of FIG. 1, the analysis method includes, for example, preparing a sample derived from an object (preparation step (S11)); and quantifying one of the target miRNA group in the sample derived from an object (quantification step (S12)).

First, the sample derived from an object is prepared (preparation step (S11)). The sample can be taken using a general method according to the type of the sample. The sample may be used as it is after collection, or may be treated so as not to inhibit a reaction for quantifying a nucleic acid or to be more suitable for the reaction. The treatment is, for example, shredding, homogenization, centrifugation, precipitation, extraction, and/or separation, and can be performed by any known means.

For example, the extraction may be performed using a commercially available nucleic acid extraction kit. As the nucleic acid extraction kit, for example, NucleoSpin (registered trademark) miRNA Plasma (manufactured by Takara Bio Inc.), Quick-cfRNA Serum & Plasma Kit (manufactured by Zymo Research Corporation), miRNeasy Serum/Plasma kit (manufactured by Qiagen), miRVana PARIS isolation kit (manufactured by Thermo Fisher Scientific Inc.), PureLink™ Total RNA Blood Kit (manufactured by Thermo Fisher Scientific Inc.), Plasma/Serum RNA Purification Kit (manufactured by Norgen Biotek Corp.), microRNA Extractor (registered trademark) SP Kit (manufactured by FUJIFILM Wako Pure Chemical Corporation), High Pure miRNA Isolation Kit (manufactured by Sigma-Aldrich), or the like can be used. Alternatively, extraction may be performed without using a commercially available kit, for example, by diluting a sample with a buffer solution, performing heat treatment at 80 to 100° ° C., centrifuging, and collecting the supernatant.

Next, one of the target miRNA group contained in the sample derived from an object is quantified (quantification step (S12)). The quantification step (S12) can be performed using a general method for quantifying RNA, particularly short-chain RNA such as miRNA. Examples of the general method include a method of reverse-transcribing a target miRNA to generate CDNA, amplifying the obtained cDNA, and detecting and quantifying an amplification product. When the RNA is a short chain, in order to facilitate amplification, it is also generally performed to extend the cDNA obtained by reverse transcription so as to add an artificial sequence to the end of the cDNA. In addition, a rolling circle amplification method is known as a technique for directly amplifying RNA in a sample without performing reverse transcription, and detecting and quantifying an amplification product. Furthermore, when the concentration of the target miRNA in the sample is relatively high or when a device capable of measuring high sensitivity can be used, directly detecting the target miRNA (or cDNA thereof) without amplifying the target miRNA is also one of general methods. Examples of the device capable of direct detection include a microarray including a nucleic acid probe that specifically binds to the target miRNA.

For the amplification, for example, a PCR method (including a qPCR method) or a LAMP method can be used. Detection and quantification may be performed after amplification or over time during amplification. For the detection and quantification, for example, a measurement method using a signal based on turbidity or absorbance, a measurement method using an optical signal, a measurement method using an electrochemical signal, a combination thereof or the like can be used. For example, the target miRNA can be quantified from the intensity or the amount of change of the signal correlated with the amount of the amplified product, the time (rise time) until the signal reaches the threshold value, or the number of rise cycles when the PCR method is used. For the detection and quantification, for example, a result of a next generation sequencing (NGS) method may be used. In that case, the target miRNA can be relatively quantified from the detection result such as the number of reads aligned with the target miRNA.

The quantitative value of target miRNA may be determined using a calibration curve representing a relationship between the detection result of the signal and the abundance of target miRNA. The calibration curve can be created by detecting signals for a plurality of standard samples containing target miRNAs at different known concentrations. The abundance of target miRNA in the sample can be calculated by comparing the calibration curve with the detection result of the signal obtained for the sample derived from an object. The abundance of target miRNA in the sample may be calculated, for example, as the number of copies of the target miRNA per unit amount of the sample.

The quantification in the quantification step (S12) may be performed using, for example, a commercially available kit. Examples of the commercially available kit include TaqMan (registered trademark) Advanced miRNA Assays (manufactured by Thermo Fisher Scientific Inc., catalog No. A25576) and miRCURY LNA (registered trademark) miRNA PCR Assays (manufactured by Qiagen, catalog No. 339306, SYBR (registered trademark) Green qPCR microRNA detection system (manufactured by Origin Technologies, Inc.), and the kit can be used together with a primer that specifically amplifies the target miRNA.

The data relating to the detection of the target miRNA obtained in the quantification step (S12) can be used for determining the presence of affection of at least any one of breast cancer, pancreatic cancer, lung cancer, gastric cancer, and colorectal cancer in the object. For example, as shown in (b) of FIG. 1, the analysis method of the first embodiment may further include a step (S13) of determining the presence of affection of at least any one of breast cancer, pancreatic cancer, lung cancer, gastric cancer, and colorectal cancer in the object, which is performed after the quantification step (S12).

It should be note that “determining the presence of affection of at least any one of breast cancer, pancreatic cancer, lung cancer, gastric cancer, and colorectal cancer in the object” means that the subject is suspected of having any one of five types of cancer but the type of cancer the subject has is not specified as any one of the five types of cancer, or the subject is not determined to have any of the five types of cancer.

In other words, “the subject is suspected of having any one of five types of cancer but the type of cancer the subject has is not specified as any one of the five types of cancer” means that the probability of the morbidity in the subject is indicated for all of the five types of cancer.

In other words, by “determining the presence of affection of at least any one of breast cancer, pancreatic cancer, lung cancer, gastric cancer, and colorectal cancer in the object”, the possibility that the subject has breast cancer, the possibility that the subject has pancreatic cancer, the possibility that the subject has lung cancer, the possibility that the subject has gastric cancer, and the possibility that the subject has colorectal cancer are presented simultaneously, or the possibility that the subject has none of the breast, pancreatic, lung, gastric and colorectal cancer is presented.

That is, this method intends to present as many possible cancer types as possible without missing any possibility of cancer, as primary screening in cancer health checkups, in view of the importance of early detection and early treatment of cancer.

Hereinafter, similar expressions excluding some cancer types from the five cancer types are used, for example, “determining whether or not the subject has at least one of the breast cancer, pancreatic cancer, lung cancer and gastric cancer” or “to determine whether or not the subject has at least one of the breast cancer, pancreatic cancer and lung cancer”; all these expressions are interpreted as above with the corresponding cancer types changed. For example, “determining whether the subject has at least one of breast, pancreatic, or lung cancer” means that the subject may have any one of three types of cancer, but the type of cancer the subject has is not specified as any one of the three types, or that the subject does not have any of the three types of cancer.

In the determination step (S13), it is possible to provide information for assisting determination that the object suffers from at least any one of breast cancer, pancreatic cancer, lung cancer, gastric cancer, and colorectal cancer.

For example, the determination step (S13) is performed based on the quantitative value of target miRNA in the sample derived from a control obtained by performing quantification on the sample derived from a control in parallel with the quantification step (S12) on the sample derived from an object. That is, the method of the first embodiment includes quantifying miRNA in the sample derived from a control; comparing the quantitative value of miRNA in the object with the quantitative value of miRNA in the control to determine whether or not the object suffers from at least any one of breast cancer, pancreatic cancer, lung cancer, gastric cancer, and colorectal cancer.

The quantitative value of target miRNA in the control is a quantitative value of target miRNA obtained in advance using the same method as used in the quantification step (S12), for example, for a sample same as or similar to the sample derived from an object (for example, when the sample derived from an object is serum, the sample derived from a control is serum or plasma). A plurality of specimens corresponding to a control may be prepared, and determination may be performed based on a numerical range including values obtained by quantifying each of the plurality of specimens.

The control may be, for example, a healthy subject. The healthy subject refers to an individual who does not suffer from at least cancer. The healthy subject is preferably a healthy individual who does not have a disease or abnormality.

The individual selected as a control may be an individual different from the object to be analyzed by the present method, but is preferably an individual belonging to the same species, that is, a human if the object is a human. In addition, physical conditions such as age, sex, height, and weight, or the number of persons of the control are not particularly limited, but the physical conditions are preferably the same as or similar to those of the object to be tested by the present analysis method.

Alternatively, the determination step (S13) may be performed based on a preset threshold value or the like. The threshold value is, for example, an abundance of target miRNA that can separate a quantitative value in a sample known to suffer from at least any one of breast cancer, pancreatic cancer, lung cancer, gastric cancer, and colorectal cancer (hereinafter, referred to as “standard sample”) from a quantitative value in a healthy subject. The standard sample is, for example, a sample of another site derived from an object, a sample derived from an individual similar to (for example, the same type as) the object, or a sample containing established cells of at least any one of breast cancer, pancreatic cancer, lung cancer, gastric cancer, and colorectal cancer. The quantitative value in the standard sample is preferably obtained by applying the same method as the quantification step (S12) of the object. The value for separating the quantitative value in the standard sample and the quantitative value in the healthy subject may be determined based on statistical criteria, the maximum value or the minimum value of the quantitative value in the standard sample may be used as the threshold value, or the maximum value or the minimum value of the quantitative value in the control may be used as the threshold value. Furthermore, the threshold value may be determined according to the quantification method, type of sample and measurement conditions used, and the like.

Alternatively, the threshold value may be determined for each object. For example, if the quantitative value of target miRNA in a healthy state of the object is monitored (for example, periodic medical examination or the like), by using the quantitative value as the threshold value, it is possible to issue an alarm indicating that there is a possibility of suffering from breast cancer, pancreatic cancer, lung cancer, gastric cancer, or colorectal cancer when the quantitative value is higher or lower than the threshold value. The threshold value may vary from individual to individual. For example, in object A in which the quantitative value of target miRNA has been usually remained at about 103 copies, once the quantitative value of target miRNA is 104 copies, it can be determined that there is a possibility of breast cancer, pancreatic cancer, lung cancer, gastric cancer, or colorectal cancer. On the other hand, in the object B in which the quantitative value of target miRNA has been remained at about 102 copies, once the quantitative value of target miRNA is 103 copies, it can be determined that there is a possibility of breast cancer, pancreatic cancer, lung cancer, gastric cancer, or colorectal cancer.

Here, the quantitative value or threshold value in the control, which serves as a criterion for determination, may be determined from past knowledge such as literature. Determination of affection also includes a high possibility of affection. Conversely, determination of no affection also includes a low possibility of affection.

In an object suffering from at least any one of breast cancer, pancreatic cancer, lung cancer, gastric cancer, and colorectal cancer, the target miRNA group includes those having a higher expression level (high expression target miRNA) and those having a lower expression level (low expression target miRNA) than the control. When the quantitative value of the low expression target miRNA is lower than that of the control, it can be determined to suffer from at least any one of breast cancer, pancreatic cancer, lung cancer, gastric cancer, and colorectal cancer. On the other hand, when the quantitative value of the high expression target miRNA is higher than that of the control, it can be determined to suffer from at least any one of breast cancer, pancreatic cancer, lung cancer, gastric cancer, and colorectal cancer.

As used herein, the phrase “the object suffers from at least any one of breast cancer, pancreatic cancer, lung cancer, gastric cancer, and colorectal cancer” includes that whether it is determined to suffer from breast cancer, pancreatic cancer, lung cancer, gastric cancer or colorectal cancer, whether it is determined to suffer from breast cancer, pancreatic cancer, lung cancer or colorectal cancer, whether it is determined to suffer from breast cancer, pancreatic cancer, lung cancer or gastric cancer, whether it is determined to suffer from breast cancer, pancreatic cancer, gastric cancer or colorectal cancer, whether it is determined to suffer from pancreatic cancer, lung cancer, gastric cancer or colorectal cancer, whether it is determined to suffer from breast cancer, lung cancer, gastric cancer or colorectal cancer, whether it is determined to suffer from breast cancer, pancreatic cancer or lung cancer, whether it is determined to suffer from breast cancer, pancreatic cancer or gastric cancer, whether it is determined to suffer from breast cancer, pancreatic cancer or colorectal cancer, whether it is determined to suffer from pancreatic cancer, lung cancer or colorectal cancer, whether it is determined to suffer from pancreatic cancer, lung cancer or gastric cancer, whether it is determined to suffer from breast cancer, lung cancer or gastric cancer, whether it is determined to suffer from breast cancer, lung cancer or colorectal cancer, whether it is determined to suffer from breast cancer, lung cancer or gastric cancer, whether it is determined to suffer from breast cancer, gastric cancer or colorectal cancer, whether it is determined to suffer from lung cancer, gastric cancer or colorectal cancer, whether it is determined to suffer from breast cancer or pancreatic cancer, whether it is determined to suffer from breast cancer or colorectal cancer, whether it is determined to suffer from breast cancer or lung cancer, whether it is determined to suffer from breast cancer or gastric cancer, whether it is determined to suffer from pancreatic cancer or lung cancer, whether it is determined to suffer from pancreatic cancer or gastric cancer, whether it is determined to suffer from pancreatic cancer or colorectal cancer, whether it is determined to suffer from lung cancer or gastric cancer, whether it is determined to suffer from lung cancer or colorectal cancer, whether it is determined to suffer from gastric cancer or colorectal cancer, whether it is determined to suffer from breast cancer, whether it is determined to suffer from pancreatic cancer, whether it is determined to suffer from lung cancer, whether it is determined to suffer from gastric cancer, and whether it is determined to suffer from colorectal cancer.

In detail, hsa-miR-30e-5p (SEQ ID NO: 9), hsa-miR-106b-5p (SEQ ID NO: 11), hsa-miR-3613-5p (SEQ ID NO: 1), hsa-miR-574-3p (SEQ ID NO: 2), hsa-miR-223-5p (SEQ ID NO: 4), hsa-miR-324-3p (SEQ ID NO: 8), hsa-miR-193a-5p (SEQ ID NO: 10), hsa-miR-4433a-3p (SEQ ID NO: 12), hsa-miR-29c-3p (SEQ ID NO: 13), hsa-miR-190a-5p (SEQ ID NO: 14), hsa-miR-194-5p (SEQ ID NO: 16), hsa-miR-29a-3p (SEQ ID NO: 17), hsa-miR-142-5p (SEQ ID NO: 18), hsa-miR-142-3p (SEQ ID NO: 20), hsa-miR-34a-5p (SEQ ID NO: 22) and hsa-miR-885-5p (SEQ ID NO: 15) can determine that the object suffers from breast cancer, pancreatic cancer, lung cancer, gastric cancer or colorectal cancer when the control is a healthy subject and the quantitative value of the control is larger than the quantitative value of the object. The hsa-miR-125b-5p (SEQ ID NO: 3) can determine that the object suffers from breast cancer, pancreatic cancer, lung cancer or colorectal cancer when the control is a healthy subject and the quantitative value of the control is larger than the quantitative value of the object. The hsa-miR-3613-3p (SEQ ID NO: 5) and the hsa-miR-941 (SEQ ID NO: 7) can determine that the object suffers from breast cancer, pancreatic cancer, lung cancer or gastric cancer when the control is a healthy subject and the quantitative value of the control is larger than the quantitative value of the object. The hsa-miR-205-5p (SEQ ID NO: 6) can determine that the object suffers from breast cancer, pancreatic cancer, gastric cancer or colorectal cancer when the control is a healthy subject and the quantitative value of the control is larger than the quantitative value of the object. The hsa-miR-483-5p (SEQ ID NO: 19) and the hsa-miR-122-5p (SEQ ID NO: 21) can determine that the object suffers from breast cancer, lung cancer or colorectal cancer when the control is a healthy subject and the quantitative value of the control is larger than the quantitative value of the object. The hsa-miR-375-3p (SEQ ID NO: 23) can determine that the object suffers from pancreatic cancer or gastric cancer when the control is a healthy subject and the quantitative value of the control is larger than the quantitative value of the object.

The difference between the quantitative value of each miRNA in the control and the quantitative value of each miRNA in the object is preferably statistically significant. Whether or not it is statistically significant can be determined by preparing a plurality of samples known to suffer from various cancers, confirming the possible numerical range of the quantitative value of each miRNA in the sample, and calculating the probability distribution in advance or calculating the probability distribution from information such as known literature.

According to a further embodiment, determining to suffer from at least any one of breast cancer, pancreatic cancer, lung cancer, gastric cancer, and colorectal cancer includes determining prognosis or recurrence of at least any one of breast cancer, pancreatic cancer, lung cancer, gastric cancer, and colorectal cancer in the object. For example, as shown in (c) of FIG. 1, the analysis method includes, after the quantification step (S12), a prognosis and recurrence determination step (S14) of determining prognosis or the presence of recurrence of at least any one of breast cancer, pancreatic cancer, lung cancer, gastric cancer, and colorectal cancer in the object from the result of quantification. In the prognosis and recurrence determination step (S14), for example, when the quantitative value is high or low, it is possible to determine that the prognosis of at least any one of breast cancer, pancreatic cancer, lung cancer, gastric cancer, and colorectal cancer in the object is poor, or at least any one of breast cancer, pancreatic cancer, lung cancer, gastric cancer, and colorectal cancer has recurred, or the possibility of recurrence is high. The quantitative value and threshold value in the control can also be used for determination of prognosis and recurrence. In particular, it may be preferable to use a threshold value determined for each object.

In addition, after the determination step (S13) and/or the prognosis and recurrence determination step (S14), it is also possible to select the type of therapy or the type of drug to be applied to the object according to the determination result and to assist the selection. For example, as shown in (d) of FIG. 1, the analysis method includes, after the determination step (S13) and/or the prognosis and recurrence determination step (S14), a selection step (S15) of selecting the type of therapy or the type of drug to be applied to the object from the determination result. Here, the therapy or drug is for the treatment of breast cancer, pancreatic cancer, lung cancer, gastric cancer or colorectal cancer. The type of therapy or the type of drug includes the used amount, timing or duration of the therapy or drug.

According to the analysis method of the first embodiment described above, it is possible to easily determine the presence of affection of breast cancer, pancreatic cancer, lung cancer, gastric cancer or colorectal cancer in an object by quantifying one target miRNA out of 23 miRNAs in the sample derived from an object and comparing the quantitative value with a quantitative value of the miRNA in the sample derived from a control. In other words, according to the present method, it is possible to easily distinguish between an object suffering from at least any one of breast cancer, pancreatic cancer, lung cancer, gastric cancer, and colorectal cancer and an object not suffering from any of at least any one of breast cancer, pancreatic cancer, lung cancer, gastric cancer, and colorectal cancer.

Since the method of the present embodiment can use serum or plasma that can be easily collected in a medical examination or the like, at least any one of breast cancer, pancreatic cancer, lung cancer, gastric cancer, and colorectal cancer can be found at an early stage. By using serum, plasma, or the like, the physical and economic burden on the object can be greatly reduced as compared with cytodiagnosis or the like, and the procedure is easy, so that the burden on an examiner is also small. In addition, since the concentration of miRNA contained in serum or plasma is stable, it is possible to perform a more accurate test.

According to a further embodiment, there is also provided an analysis method for assisting determination of the presence of affection of at least any one of breast cancer, pancreatic cancer, lung cancer, gastric cancer, and colorectal cancer in an object, the analysis method including quantifying a target miRNA in the sample derived from an object (quantification step (S12)). The phrase “assisting determination” includes, for example, acquiring information about the possibility that the object suffers from at least any one of breast cancer, pancreatic cancer, lung cancer, gastric cancer, and colorectal cancer. The term “information” is, for example, information about an analysis result of a sample, and can be, for example, a quantitative value. According to the present method, it is possible to acquire more accurate information for determining the presence of affection, determining prognosis, determining the presence of recurrence, selecting the therapy or drug to be applied to the object of at least any one of breast cancer, pancreatic cancer, lung cancer, gastric cancer, and colorectal cancer in the object, or the like.

According to a further embodiment, the control may be, for example, an individual confirmed to suffer from any one of breast cancer, pancreatic cancer, lung cancer, gastric cancer, and colorectal cancer by examination or the like. That is, the control is an individual whether or not suffering from any one of breast cancer, pancreatic cancer, lung cancer, gastric cancer, and colorectal cancer is known. That is, in such a determination step (S13), whether or not the object suffers from the cancer type from which the control suffers is determined. Specifically, the determination is made by quantifying a biomarker in the sample derived from an object with reference to a quantitative value of the biomarker indicating that the object suffers from a specific cancer in the sample derived from a control, and comparing the biomarker with the reference.

In detail, each of the hsa-miR-30e-5p (SEQ ID NO: 9), the hsa-miR-3613-5p (SEQ ID NO: 1), the hsa-miR-223-5p (SEQ ID NO: 4), the hsa-miR-3613-3p (SEQ ID NO: 5), the hsa-miR-941 (SEQ ID NO: 7), the hsa-miR-324-3p (SEQ ID NO: 8), the hsa-miR-29c-3p (SEQ ID NO: 13), the hsa-miR-190a-5p (SEQ ID NO: 14) and the hsa-miR-142-3p (SEQ ID NO: 20) can determine that the object suffers from breast cancer, pancreatic cancer, lung cancer or gastric cancer when the control is a colorectal cancer subject and the quantitative value of the control is larger than the quantitative value of the object. Each of the hsa-miR-574-3p (SEQ ID NO: 2) and the hsa-miR-29a-3p (SEQ ID NO: 17) can determine that the object suffers from breast cancer, pancreatic cancer or lung cancer when the control is a colorectal cancer subject and the quantitative value of the control is larger than the quantitative value of the object. Each of the hsa-miR-205-5p (SEQ ID NO: 6) can determine that the object suffers from lung cancer when the control is a colorectal cancer subject and the quantitative value of the control is smaller than the quantitative value of the object. Each of the hsa-miR-193a-5p (SEQ ID NO: 10) can determine that the object suffers from breast cancer when the control is a colorectal cancer subject and the quantitative value of the control is larger than the quantitative value of the object. Each of the hsa-miR-106b-5p (SEQ ID NO: 11) and the hsa-miR-142-5p (SEQ ID NO: 18) can determine that the object suffers from breast cancer, pancreatic cancer or gastric cancer when the control is a colorectal cancer subject and the quantitative value of the control is larger than the quantitative value of the object. The hsa-miR-4433a-3p (SEQ ID NO: 12) can determine that the object suffers from breast cancer or pancreatic cancer when the control is a colorectal cancer subject and the quantitative value of the control is larger than the quantitative value of the object.

Note that the hsa-miR-205-5p (SEQ ID NO: 6), the hsa-miR-223-5p (SEQ ID NO: 4), the hsa-miR-3613-3p (SEQ ID NO: 5), the hsa-miR-324-3p (SEQ ID NO: 8) and the hsa-miR-4433a-3p (SEQ ID NO: 12) can determine that the object suffers from lung cancer when the control is a breast cancer subject and the quantitative value of the control is smaller than the quantitative value of the object. The hsa-miR-29c-3p (SEQ ID NO: 13), the hsa-miR-574-3p (SEQ ID NO: 2), the hsa-miR-125b-5p (SEQ ID NO: 3) and the hsa-miR-122-5p (SEQ ID NO: 21) can determine that the object suffers from gastric cancer when the control is a breast cancer subject and the quantitative value of the control is smaller than the quantitative value of the object. The hsa-miR-34a-5p (SEQ ID NO: 22) can determine that the object suffers from pancreatic cancer or gastric cancer when the control is a breast cancer subject and the quantitative value of the control is smaller than the quantitative value of the object. The hsa-miR-205-5p (SEQ ID NO: 6) can determine that the object suffers from pancreatic cancer or gastric cancer when the control is a lung cancer subject and the quantitative value of the control is larger than the quantitative value of the object. The hsa-miR-122-5p (SEQ ID NO: 21) can determine that the object suffers from gastric cancer when the control is a lung cancer subject and the quantitative value of the control is smaller than the quantitative value of the object.

According to a further embodiment, the present analysis method can also be used for detection of breast cancer cells, pancreatic cancer cells, lung cancer cells, gastric cancer cells or colorectal cancer cells in a sample not derived from an object, and the like. For example, in the case of artificially producing breast cancer cells, pancreatic cancer cells, lung cancer cells, gastric cancer cells or colorectal cancer cells, it can also be used when confirming whether or not the same cells are present in the produced cell-containing solution, and the like.

### (Marker)

According to the first embodiment, there is provided a marker for detecting at least any one of breast cancer, pancreatic cancer, lung cancer, gastric cancer, and colorectal cancer of the object, the marker including one target miRNA.

Here, the “marker” refers to a substance capable of determining whether or not a sample and/or an object from which the sample is derived is in a specific state by detecting its presence or concentration in the sample.

As the marker for detecting at least any one of breast cancer, pancreatic cancer, lung cancer, gastric cancer, and colorectal cancer of the first embodiment, for example, by measuring the abundance (quantitative value) of the marker in the sample derived from an object, it is possible to determine the presence of affection of at least any one of breast cancer, pancreatic cancer, lung cancer, gastric cancer, and colorectal cancer in the object, determine prognosis or the presence of recurrence, select the therapy or drug to be applied to the object, or the like, as described above.

### (Kit)

The kit contains reagents that can be used in a general method for quantifying RNA, particularly short-chain RNA such as miRNA, and a nucleic acid capable of specifically binding to a target miRNA (that is, it hybridizes with the target miRNA). When a qPCR method is used for detection of the target miRNA, the nucleic acid capable of specifically binding to the target miRNA may be a reverse transcription (RT) primer for reverse transcription of the target miRNA, an elongation (EL) primer for elongation of the target miRNA, or an amplification primer set for amplification of the target miRNA.

The RT primer is a primer for obtaining cDNA of the target miRNA. The RT primer includes a sequence complementary to at least a portion of the sequence of the target miRNA. The RT primer may further include an artificial sequence added to the cDNA to facilitate amplification of the cDNA of the target miRNA.

The RT primer is a primer for obtaining cDNA of the target miRNA. The RT primer includes a sequence complementary to at least a portion of the sequence of the target miRNA. The RT primer may further include an artificial sequence added to the cDNA to facilitate amplification of the cDNA of the target miRNA.

The EL primer is a primer for adding an artificial sequence to the cDNA of the target miRNA to facilitate amplification of the cDNA. The EL primer may include a sequence complementary to at least a portion of the sequence of the cDNA of the target miRNA and a sequence added for elongation of each cDNA.

The amplification primer set contains at least a forward primer and a reverse primer, for example, for PCR method. Alternatively, the amplification primer set may be for LAMP method, and may contain primers of a sequence corresponding to the base sequence of the target miRNA used in the general LAMP method. Alternatively, the amplification primer set may be for NGS method, for example, and may contain a forward primer containing an artificial adaptor sequence and a reverse primer containing a complementary sequence thereof. The amplification primer set for NGS method may contain a plurality of types of combinations of forward primers and reverse primers including different barcode sequences in order to simultaneously analyze a plurality of specimens. When the amplification primer set is used in rolling cycle amplification method, the kit further contains a circular single-stranded DNA that is hybridized by the amplification primer and serves as a template for amplification.

Each primer contained in the amplification primer set may be designed to bind to cDNA of the target miRNA or a complementary sequence thereof, or may be designed to bind to an artificial sequence added by an RT primer and/or an EL primer.

Further, when the target miRNA in the sample is directly detected by a microarray, the nucleic acid capable of specifically binding to the target miRNA is a nucleic acid probe included in the microarray. The nucleic acid probe may have at least a portion of, or a complementary sequence of, the sequence of the target miRNA, cDNA thereof, or an amplification product thereof.

The nucleic acid contained in the kit may be provided by being stored in a container together with an appropriate carrier individually or in combination. The appropriate carrier is, for example, water, a physiological solution or a buffer. The container is, for example, a tube or a microtiter plate. Alternatively, these nucleic acids may be provided by being immobilized on a solid phase such as a microfluidic chip.

In addition to the nucleic acid, the kit may contain a reagent used for reverse transcription, elongation or amplification, for example, an enzyme, a substrate, and/or a labeling substance that generates an optical signal or an electrochemical signal used for detection, and the like. The labeling substance is, for example, a fluorescent dye such as SYBR Green, EvaGreen (registered trademark), or SYTO (registered trademark) 82, or an indicator such as a metal complex such as ruthenium hexamine in the case of current detection.

The kit can be used, for example, for determining the presence of affection, determining prognosis, determining the presence of recurrence, selecting the type of therapy or the type of drug of at least any one of breast cancer, pancreatic cancer, lung cancer, gastric cancer, and colorectal cancer in the object, or the like.

According to a further embodiment, a kit for detecting at least any one of breast cancer, pancreatic cancer, lung cancer, gastric cancer, and colorectal cancer is provided as a composition or a diagnostic agent for diagnosing at least any one of breast cancer, pancreatic cancer, lung cancer, gastric cancer, and colorectal cancer. Further, according to the embodiment, there is also provided a diagnostic composition of at least any one of breast cancer, pancreatic cancer, lung cancer, gastric cancer, and colorectal cancer, or a use of at least any one of the nucleic acids in the production of a diagnostic agent of at least any one of breast cancer, pancreatic cancer, lung cancer, gastric cancer, and colorectal cancer.

### Second Embodiment

### (Analysis Method)

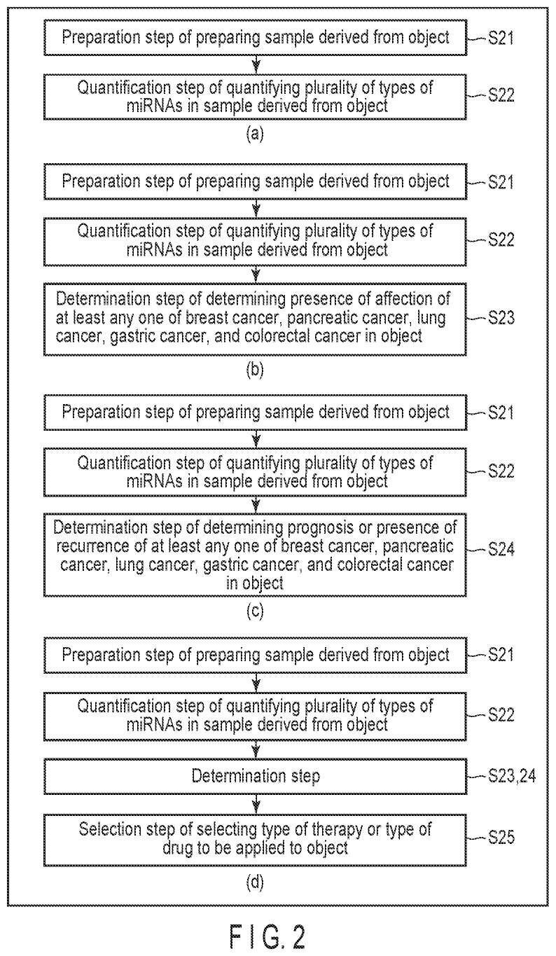

As shown in of (a) of FIG. 2, the method of the second embodiment includes, for example, preparing a sample derived from an object (preparation step (S21)); and quantifying a plurality of types of target miRNAs among target miRNAs of hsa-miR-205-5p, hsa-miR-30e-5p, hsa-miR-106b-5p, hsa-miR-3613-5p, hsa-miR-483-5p, hsa-miR-574-3p, hsa-miR-125b-5p, hsa-miR-223-5p, hsa-miR-3613-3p, hsa-miR-941, hsa-miR-324-3p, hsa-miR-193a-5p, hsa-miR-4433a-3p, hsa-miR-29c-3p, hsa-miR-190a-5p, hsa-miR-885-5p, hsa-miR-194-5p, hsa-miR-29a-3p, hsa-miR-142-5p, hsa-miR-142-3p, hsa-miR-122-5p, hsa-miR-34a-5p, and hsa-miR-375-3p in the sample derived from an object (quantification step (S22)).

In the case of quantifying a plurality of types (that is, at least two types) of target miRNAs, a separate reaction system may be prepared for each type of target miRNA, and quantification may be performed by performing reverse transcription, extension, amplification and/or detection for each reaction system, or the plurality of types of target miRNAs may be detected and quantified in the same reaction system using a flow channel chip or the like capable of simultaneously detecting a plurality of nucleic acids. In addition, for example, by using NGS method, it is also possible to amplify, detect, and quantify the plurality of types of target miRNAs in a massively parallel manner in the same reaction system.

The data including the quantitative values of the plurality of types of target miRNAs obtained in the quantification step (S22) can be used for determining the presence of affection of at least any one of breast cancer, pancreatic cancer, lung cancer, gastric cancer, and colorectal cancer in the object or assisting the determination. For example, as shown in (b) of FIG. 2, the analysis method of the second embodiment can further include a determination step (S23) that can be performed after the quantification step (S22).

In the determination step (S23), for example, a criterion similar to that of the first embodiment is set for each target miRNA, and determination is made by comparing the criterion with the quantitative value obtained in the quantification step (S22). The criterion of each target miRNA of the object is preferably a quantitative value, a threshold value, or the like of the corresponding target miRNA of the control, and each threshold value is preferably determined for each type of target miRNA. That is, the method of the second embodiment includes quantifying a plurality of types of miRNAs in a sample derived from a control; and comparing the quantitative values of the plurality of types of miRNAs in the object with the quantitative values of the plurality of types of miRNAs in the control to determine that the object suffers from at least any one of breast cancer, pancreatic cancer, lung cancer, gastric cancer, and colorectal cancer. The control is a healthy subject or a specimen known to suffer from at least any one of breast cancer, pancreatic cancer, lung cancer, gastric cancer, and colorectal cancer.

Data of quantitative values of the plurality of types of target miRNAs is obtained by the quantification step (S22), and when all types of target miRNAs for which data of quantitative values is obtained are higher or lower than the reference, it may be determined that the object suffers from at least any one of breast cancer, pancreatic cancer, lung cancer, gastric cancer, and colorectal cancer. Alternatively, when a quantitative value of one target miRNAs of the plurality of types is higher or lower than the reference such as a threshold value, it may be determined that the object suffers from at least any one of breast cancer, pancreatic cancer, lung cancer, gastric cancer, and colorectal cancer, and when quantitative values of at least two or more of the target miRNAs are higher or lower than the reference, it may be determined that the object suffers from at least any one of breast cancer, pancreatic cancer, lung cancer, gastric cancer, and colorectal cancer. When an arithmetic value obtained by combining the quantitative values of two or more types of target miRNAs and performing four arithmetic operations, exponential and logarithmic operations, and the like is higher or lower than the reference, it may be determined that the object suffers from at least any one of breast cancer, pancreatic cancer, lung cancer, gastric cancer, and colorectal cancer.

Also, the determination may be made after quantifying (that is, modeling) the difference or relevance between the reference and the quantitative value. A determination method including modeling is, for example, the following method: preparing a learning sample derived from a subject other than the object; quantifying each of the miRNA group in the learning sample; constructing a determination algorithm for determining the presence of affection of at least one of breast cancer, pancreatic cancer, lung cancer, gastric cancer, and colorectal cancer, using information on the subject and a quantitative value of each of the miRNA group in the learning sample as model learning data; and determining the presence of affection of at least one cancer of breast cancer, pancreatic cancer, lung cancer, gastric cancer, and colorectal cancer of the object from quantitative values of miRNAs and the information on the subject by using the determination algorithm.

For example, the degree of relevance may be quantified by creating a model in which the weighting of the arithmetic value is differentiated for each of a plurality of types of miRNAs, the weighting coefficient is set to be high for a miRNA having high relevance to each cancer type, and the weighting coefficient is set to be low for a miRNA having low relevance. At this time, it may be determined that the object suffers from at least any one of breast cancer, pancreatic cancer, lung cancer, gastric cancer, and colorectal cancer if there is a certain degree of relevance as a criterion for determination.

The degree of relevance between the plurality of types of miRNAs and each cancer type may be determined by a binary logistic regression model or a multinomial logistic regression model. Examples of the variable selection method in the binary logistic regression model or the multinomial logistic regression model include sparse estimation (such as Lasso, SCAD, MCP), a stepwise method, and a brute force method (such as best subset selection). In addition, not only the binary logistic regression model or the multinomial logistic regression model but also, for example, a variable selection method by a statistical method such as ANOVA analysis or Kruskal-Wallis analysis, a variable selection method by a contribution degree and a p value, or a machine learning method may be used to determine the degree of relevance between the plurality of types of miRNAs and each cancer type.

By performing machine learning using training data prepared in advance, a learned model capable of determining affection of at least any one of breast cancer, pancreatic cancer, lung cancer, gastric cancer, and colorectal cancer can be created as a type of determination algorithm. The training data may include information on the subject. The information on the subject may be information about the presence of affection of various cancers, and may also include personal data such as previous diseases, gender, BMI, and smoking rate. Using this learned model, it may be determined whether or not the object suffers from at least any one of breast cancer, pancreatic cancer, lung cancer, gastric cancer, and colorectal cancer from data of quantitative values of a plurality of target miRNAs confirmed in the sample derived from an object.

In order to generate the learned model, it is preferable to prepare a plurality of sample data of breast cancer subjects, pancreatic cancer subjects, lung cancer subjects, gastric cancer subjects, and colorectal cancer subjects and sample data of healthy subjects. In a case where the machine learning is, for example, a neural network, the learning by the neural network sets configuration of the neural network such that the affection determination result is obtained as an output when the training data is input. In addition, the machine learning model is not limited to this method, and random forest, boosting, other neural network, or the like may be used.

The plurality of types of target miRNAs includes combinations of target miRNAs (SEQ ID NOs: 1 to 23) shown in Table 1. However, this combination is preferably a combination confirmed to be preferable as a marker by investigating the sensitivity and specificity of detection and quantification under specific conditions (for example, objects, samples, types of detection and quantification methods, and the like) in advance. The combination of target miRNAs may be a combination of a high expression target miRNA and a low expression target miRNA.

Examples of the combination of target miRNAs include a combination of target miRNAs including hsa-miR-223-5p (SEQ ID NO: 4), hsa-miR-205-5p (SEQ ID NO: 6), hsa-miR-30e-5p (SEQ ID NO: 9), hsa-miR-106b-5p (SEQ ID NO: 11), hsa-miR-29c-3p (SEQ ID NO: 13), and hsa-miR-486-5p (SEQ ID NO: 176) (combination 1). For example, in the determination step (S23) using such a combination of target miRNAs, the quantitative value of the combination of an object can be applied to a determination algorithm such as a binary logistic regression model created in advance to determine the presence of affection of colorectal cancer of the object.

The combination of target miRNAs is not limited to combination 1, and other preferable combinations have been specified. For example, when the subject is colorectal cancer, it has been confirmed that combination 2 and combination 3 shown in Table 9 (described later) are also preferable combinations. In addition, by applying the quantitative values of combination 4, combination 5, and combination 6 shown in Table 10 (described later) to the determination algorithm, the presence of affection of pancreatic cancer of the object can be determined.

According to a method of a further embodiment, a prognosis and recurrence determination step (S24) similar to that of the first embodiment is included after the quantification step (S22). Further, the method includes a selection step (S25) similar to that of the first embodiment after the determination step (S23) and/or the prognosis and recurrence determination step (S24). Here, the therapy or drug is for treating at least any one of breast cancer, pancreatic cancer, lung cancer, gastric cancer, and colorectal cancer. By the selection step (S25), for example, the type of therapy, the type of drug, the used amount, timing or duration of the therapy or drug.

### (Marker)

According to the second embodiment, there is provided a marker for detecting at least any one of breast cancer, pancreatic cancer, lung cancer, gastric cancer, and colorectal cancer, the marker including a plurality of types of target miRNAs. For example, by measuring the abundance of the marker in the sample derived from an object and obtaining a quantitative value, it is possible to determine the presence of affection of at least any one of breast cancer, pancreatic cancer, lung cancer, gastric cancer, and colorectal cancer in the object, determine prognosis or the presence of recurrence, select the therapy or drug to be applied to the object, or the like, as described above.

### (Kit)

According to the second embodiment, a kit for detecting at least any one of breast cancer, pancreatic cancer, lung cancer, gastric cancer, and colorectal cancer is provided. The kit is a kit for detecting and quantifying a plurality of types of miRNAs by a general method. As in the kit of the first embodiment, the kit contains reagents that can be used in a general method for quantifying RNA, particularly short-chain RNA such as miRNA, and a plurality of types of nucleic acids capable of specifically binding to each target miRNA.

According to the kit of the second embodiment, by detecting and quantifying a plurality of types of miRNAs, it may be possible to determine the presence of affection of breast cancer, pancreatic cancer, lung cancer, gastric cancer, or colorectal cancer with higher sensitivity depending on the combination.

### EXAMPLES

Hereinafter, an experiment performed to obtain the marker of the present embodiment and verification of a determination algorithm using the marker of the present embodiment will be described.

### Example 1. Comprehensive Search and Selection of miRNA Markers for Distinguishing Between Various Cancer Subjects and Healthy Subjects

Some miRNA markers capable of distinguishing breast cancer subjects, pancreatic cancer subjects, and lung cancer subjects from healthy subjects were searched as follows.

### Preparation of Specimens

As specimens, breast cancer patient sera, pancreatic cancer patient sera, lung cancer patient sera, and healthy person sera were prepared for 24 specimens each, for a total of 96 specimens.

Treatment of Specimens and Quantification of miRNAs

From 300 μL of all sera, miRNAs were extracted using NucleoSpin (registered trademark) miRNA Plasma (manufactured by Takara Bio Inc.).

For synthesis of cDNA, TaqMan miRNA cDNA Synthesis Kit (Applied Biosystems, Cat. A28007) was used. The TaqMan miRNA CDNA Synthesis Kit is a kit in which addition of a poly (A) chain to the 3′ end of mature RNA and ligation of an adaptor sequence to the 5′ end are performed, whereby not target-specific but all mature RNAs present in a sample are generally reverse-transcribed. Therefore, CDNAs corresponding to all mature RNAs including miRNA in each specimen were obtained.

miRNAs among the obtained cDNAs were quantified by performing RT-qPCR using TaqMan Fast Advanced Master Mix (manufactured by Applied Biosystems) and TaqMan Advanced miRNA Assays (manufactured by Applied Biosystems) according to the protocol included in the package. The TaqMan PCR method, which is one of 5′ nuclease assays, is an amplification method characterized by having excellent detection accuracy by using a primer for amplifying a target in combination with a TaqMan probe in which fluorescence resonance energy transfer (FRET) occurs in the molecule and which specifically binds to the target. The quantitative value of cDNA obtained by applying this TaqMan PCR method is, for example, the number of cycles (Ct value) until cDNA corresponding to each probe and primer reaches the detection standard.

Note that the quantitative value was corrected with a quantitative value of miRNA commonly present in serum samples of healthy subjects and various cancer subjects (hereinafter, “standard miRNA”), considering that the activity level of the entire miRNA may be different for each individual, and the yield of the extraction or amplification reaction may be different for each type of cell or specimen in the treatment of the specimen. Specifically, correction was performed using a quantitative value of hsa-miR-486-5p (SEQ ID NO: 176) known to have a high expression level in healthy subjects and various cancer subjects. The difference (ΔCt value) by subtracting the Ct value of hsa-miR-486-5p from the Ct value of each miRNA was normalized, so as to be used for correction as an index of the expression level, and the expression level ratio was determined among the groups of healthy person, breast cancer, lung cancer, and pancreatic cancer.

### Results of Search

The measurement results of RT-qPCR using the TaqMan PCR method are shown in Table 2. As shown in Table 2, a total of 175 types of miRNAs were observed. In Table 2, “−” indicates data in which amplification by TaqMan PCR method was not sufficiently observed and the detection reference value was not reached in healthy subjects, breast cancer subjects, pancreatic cancer subjects, and lung cancer subjects.

TABLE 2-1

| | | Ratio to healthy subject | | |
|---|---|---|---|---|
| Sequence | | Breast | Pancreatic | Lung |
| number | Name of miRNA | cancer | cancer | cancer |
| 1 | hsa-miR-3613-5p | 2.77 | 9.69 | 34.14 |
| 2 | hsa-miR-574-3p | 1.71 | 1.71 | 1.30 |
| 3 | hsa-miR-125b-5p | 0.14 | 2.61 | 5.98 |
| 4 | hsa-miR-223-5p | 0.18 | 0.56 | 1.07 |
| 5 | hsa-miR-3613-3p | 0.32 | 0.64 | 0.86 |
| 6 | hsa-miR-205-5p | 0.28 | 0.27 | 0.73 |
| 7 | hsa-miR-941 | 0.17 | 0.44 | 0.69 |
| 8 | hsa-miR-324-3p | 0.49 | 0.53 | 0.66 |
| 9 | hsa-miR-30e-5p | 0.05 | 0.23 | 0.56 |
| 10 | hsa-miR-193a-5p | 0.34 | 0.48 | 0.53 |
| 11 | hsa-miR-106b-5p | 0.13 | 0.13 | 0.42 |
| 12 | hsa-miR-4433a-3p | 0.23 | 0.54 | 0.45 |
| 13 | hsa-miR-29c-3p | 0.17 | 0.29 | 0.36 |
| 14 | hsa-miR-190a-5p | 0.17 | 0.26 | 0.41 |
| 15 | hsa-miR-885-5p | 0.21 | 0.21 | 0.66 |
| 16 | hsa-miR-194-5p | 0.15 | 0.50 | 0.35 |
| 17 | hsa-miR-29a-3p | 0.14 | 0.28 | 0.32 |
| 18 | hsa-miR-142-5p | 0.11 | 0.22 | 0.31 |
| 19 | hsa-miR-483-5p | 0.23 | 0.60 | 0.26 |
| 20 | hsa-miR-142-3p | 0.15 | 0.16 | 0.24 |
| 21 | hsa-miR-122-5p | 0.12 | 0.98 | 0.23 |
| 22 | hsa-miR-34a-5p | 0.07 | 0.54 | 0.25 |
| 23 | hsa-miR-375-3p | 0.05 | 0.05 | 0.28 |
| 24 | hsa-miR-30d-5p | 0.40 | 0.61 | 0.75 |
| 25 | hsa-miR-451a | 0.18 | 0.27 | 0.34 |
| 26 | hsa-miR-148a-3p | 0.24 | 0.56 | 0.57 |
| 27 | hsa-miR-103a-3p | 0.28 | 0.38 | 0.43 |
| 28 | hsa-miR-484 | 0.68 | 0.85 | 1.00 |
| 29 | hsa-miR-584-5p | 0.42 | 0.49 | 0.52 |
| 30 | hsa-miR-107 | 0.30 | 0.47 | 0.46 |
| 31 | hsa-miR-101-3p | 0.30 | 0.35 | 0.47 |
| 32 | hsa-miR-143-3p | 0.19 | 0.41 | 0.66 |
| 33 | hsa-miR-382-5p | 0.36 | 0.30 | 0.40 |
| 34 | hsa-miR-626 | — | — | — |
| 35 | hsa-miR-432-5p | 1.37 | 0.56 | 1.35 |
| 36 | hsa-miR-4501 | — | — | — |
| 37 | hsa-miR-22-3p | 0.21 | 0.32 | 0.31 |
| 38 | hsa-miR-27b-3p | 0.33 | 0.50 | 0.72 |
| 39 | hsa-miR-6785-5p | 0.26 | 0.27 | 0.76 |
| 40 | hsa-miR-4433b-5p | 1.39 | 1.17 | 1.54 |
| 41 | hsa-miR-7847-3p | — | — | — |
| 42 | hsa-miR-128-3p | 0.56 | 0.91 | 1.11 |
| 43 | hsa-miR-10b-5p | 0.55 | 0.67 | 0.88 |
| 44 | hsa-miR-5007-3p | 0.29 | 0.31 | 0.31 |
| 45 | hsa-miR-6728-5p | — | — | — |
| 46 | hsa-miR-155-5p | 0.29 | 0.40 | 0.51 |
| 47 | hsa-miR-99b-5p | 0.46 | 0.44 | 0.54 |
| 48 | hsa-miR-10b-3p | 0.37 | 0.44 | 1.12 |
| 49 | hsa-miR-2115-3p | — | — | — |
| 50 | hsa-miR-4782-5p | — | — | — |
| 51 | hsa-miR-664a-3p | 0.58 | 0.70 | 0.92 |
| 52 | hsa-miR-222-3p | 0.30 | 0.46 | 0.54 |
| 53 | hsa-miR-5189-3p | 0.73 | 0.20 | 0.54 |
| 54 | hsa-miR-6855-3p | 0.06 | 0.04 | 0.54 |
| 55 | hsa-miR-20a-5p | 0.40 | 0.67 | 0.80 |
| 56 | hsa-miR-4798-5p | 0.02 | 0.01 | 0.01 |
| 57 | hsa-miR-98-5p | 0.37 | 0.68 | 0.87 |
| 58 | hsa-miR-18a-5p | 0.32 | 0.51 | 0.57 |
| 59 | hsa-miR-6749-5p | 0.39 | 0.49 | 0.51 |
| 60 | hsa-miR-4254 | — | — | — |
| 61 | hsa-miR-518a-3p | 1.65 | 0.53 | 0.30 |
| 62 | hsa-miR-326 | 0.72 | 0.73 | 0.88 |
| 63 | hsa-miR-4440 | 0.64 | 1.01 | 0.85 |
| 64 | hsa-miR-19b-3p | 0.21 | 0.39 | 0.47 |
| 65 | hsa-miR-885-3p | 0.17 | 0.22 | 0.32 |
| 66 | hsa-miR-483-3p | 0.35 | 0.70 | 0.48 |
| 67 | hsa-miR-2276-3p | 0.30 | 0.78 | 0.85 |
| 68 | hsa-miR-181b-5p | 0.38 | 0.55 | 0.60 |
| 69 | hsa-miR-196a-5p | 0.23 | 0.29 | 0.38 |
| 70 | hsa-miR-4322 | 0.76 | 0.19 | 0.39 |
| 71 | hsa-miR-4780 | — | — | — |
| 72 | hsa-miR-668-3p | — | — | — |
| 73 | hsa-miR-196b-5p | 0.54 | 0.78 | 1.14 |
| 74 | hsa-miR-17-5p | 0.37 | 0.65 | 0.74 |
| 75 | hsa-miR-1306-5p | 1.06 | 1.99 | 5.72 |
| 76 | hsa-miR-653-3p | 0.13 | 4.90 | 2.67 |
| 77 | hsa-miR-4502 | — | — | — |
| 78 | hsa-miR-3609 | — | 9.45 | 4.83 |
| 79 | hsa-miR-3126-3p | — | — | 0.42 |
| 80 | hsa-miR-514a-5p | 0.23 | 0.49 | 0.72 |
| 81 | hsa-miR-3662 | — | — | — |
| 82 | hsa-miR-8087 | 0.21 | 0.19 | 0.51 |
| 83 | hsa-miR-133a-3p | 0.42 | 1.16 | 1.03 |
| 84 | hsa-miR-4540 | — | — | — |
| 85 | hsa-miR-203a-3p | — | — | — |
| 86 | hsa-miR-3666 | — | — | — |
| 87 | hsa-miR-4803 | — | 0.00 | — |
| 88 | hsa-miR-138-1-3p | 0.02 | 0.14 | 0.18 |
| 89 | hsa-miR-5700 | 0.17 | 0.14 | 0.24 |
| 90 | hsa-miR-22-5p | 0.21 | 0.44 | 0.47 |
| 91 | hsa-miR-5695 | — | 1.32 | 1.39 |
| 92 | hsa-miR-1-3p | 0.34 | 0.52 | 0.59 |
| 93 | hsa-miR-365a-5p | 0.83 | 0.31 | 0.71 |
| 94 | hsa-miR-6811-5p | — | — | — |
| 95 | hsa-miR-4661-5p | — | — | — |
| 96 | hsa-miR-574-5p | 0.23 | 0.31 | 0.42 |
| 97 | hsa-miR-505-5p | 0.38 | 1.03 | 1.01 |
| 98 | hsa-miR-4771 | — | — | — |
| 99 | hsa-miR-3163 | — | 0.01 | 0.03 |
| 100 | hsa-miR-31-5p | 0.21 | 0.20 | 0.26 |
| 101 | hsa-miR-20a-3p | 0.53 | 0.27 | 0.47 |
| 102 | hsa-miR-4777-3p | 1.11 | 0.64 | 0.43 |
| 103 | hsa-miR-552-5p | 0.41 | 0.45 | 0.52 |
| 104 | hsa-miR-3941 | — | — | — |
| 105 | hsa-miR-6781-3p | — | — | — |
| 106 | hsa-miR-550a-5p | 0.43 | 0.51 | 0.61 |
| 107 | hsa-miR-16-5p | 0.35 | 0.68 | 0.79 |
| 108 | hsa-let-7b-5p | 0.30 | 0.55 | 0.72 |
| 109 | hsa-miR-423-5p | 0.41 | 0.76 | 0.79 |
| 110 | hsa-let-7a-5p | 0.34 | 0.57 | 0.69 |
| 111 | hsa-let-71-5p | 0.30 | 0.52 | 0.54 |
| 112 | hsa-miR-486-3p | 0.56 | 0.84 | 0.99 |
| 113 | hsa-let-7f-5p | 0.32 | 0.49 | 0.61 |
| 114 | hsa-miR-126-3p | 0.30 | 0.46 | 0.50 |
| 115 | hsa-miR-25-3p | 0.41 | 0.78 | 0.89 |
| 116 | hsa-miR-146a-5p | 0.33 | 0.55 | 0.58 |
| 117 | hsa-miR-21-5p | 0.21 | 0.36 | 0.44 |
| 118 | hsa-miR-425-5p | 0.35 | 0.60 | 0.71 |
| 119 | hsa-miR-93-5p | 0.35 | 0.64 | 0.76 |
| 120 | hsa-miR-150-5p | 0.47 | 0.51 | 0.60 |
| 121 | hsa-miR-191-5p | 0.31 | 0.57 | 0.70 |
| 122 | hsa-miR-125a-5p | 0.50 | 0.66 | 0.77 |
| 123 | hsa-miR-342-3p | 0.51 | 0.70 | 0.92 |
| 124 | hsa-miR-221-3p | 0.31 | 0.57 | 0.66 |
| 125 | hsa-miR-26b-5p | 0.22 | 0.39 | 0.54 |
| 126 | hsa-miR-23a-3p | 0.29 | 0.58 | 0.80 |
| 127 | hsa-miR-197-3p | 0.43 | 0.63 | 1.04 |
| 128 | hsa-miR-24-3p | 0.26 | 0.55 | 0.69 |
| 129 | hsa-let-7d-3p | 0.47 | 0.78 | 1.08 |
| 130 | hsa-miR-328-3p | 0.47 | 0.58 | 0.75 |
| 131 | hsa-miR-320b | 0.46 | 0.56 | 0.55 |
| 132 | hsa-miR-3130-5p | — | — | — |
| 133 | hsa-miR-744-5p | 0.21 | 0.51 | 0.59 |
| 134 | hsa-miR-889-5p | — | — | — |
| 135 | hsa-miR-625-3p | 0.26 | 1.04 | 1.58 |
| 136 | hsa-miR-660-5p | 0.22 | 0.41 | 0.44 |
| 137 | hsa-let-7e-5p | 0.27 | 0.39 | 0.47 |
| 138 | hsa-miR-181a-5p | 0.20 | 0.46 | 0.47 |
| 139 | hsa-miR-28-3p | 0.27 | 0.53 | 0.66 |
| 140 | hsa-miR-4732-5p | 1.12 | 1.36 | 1.20 |
| 141 | hsa-miR-139-3p | — | — | — |
| 142 | hsa-miR-324-5p | 0.23 | 0.45 | 0.53 |
| 143 | hsa-miR-127-3p | 0.23 | 0.23 | 0.30 |
| 144 | hsa-miR-16-2-3p | 0.30 | 0.55 | 0.64 |
| 145 | hsa-miR-186-5p | 0.22 | 0.48 | 0.59 |
| 146 | hsa-miR-6797-5p | — | — | — |
| 147 | hsa-miR-27a-3p | 0.20 | 0.58 | 0.84 |
| 148 | hsa-miR-425-3p | 0.32 | 0.60 | 0.73 |
| 149 | hsa-miR-532-5p | 0.26 | 0.56 | 0.59 |
| 150 | hsa-miR-320a-3p | 0.42 | 0.42 | 0.69 |
| 151 | hsa-miR-3178 | 0.81 | 0.81 | 1.76 |
| 152 | hsa-miR-361-5p | 0.36 | 0.36 | 0.72 |
| 153 | hsa-miR-375-5p | — | — | — |
| 154 | hsa-miR-148a-5p | 0.95 | 0.95 | 0.14 |
| 155 | hsa-miR-6738-5p | — | — | — |
| 156 | hsa-let-7g-5p | 0.26 | 0.26 | 0.42 |
| 157 | hsa-let-7b-3p | 0.40 | 0.40 | 0.81 |
| 158 | hsa-miR-6794-3p | — | — | — |
| 159 | hsa-miR-4302 | 0.30 | 0.30 | 0.85 |
| 160 | hsa-miR-5586-5p | — | — | — |
| 161 | hsa-miR-629-5p | 0.30 | 0.30 | 0.62 |
| 162 | hsa-miR-378a-3p | 0.20 | 0.20 | 0.59 |
| 163 | hsa-miR-606 | 0.32 | 0.32 | 1.00 |
| 164 | hsa-miR-92b-3p | 0.67 | 0.67 | 1.01 |
| 165 | hsa-miR-4778-5p | — | — | — |
| 166 | hsa-miR-3617-3p | — | — | — |
| 167 | hsa-miR-8060 | — | — | — |
| 168 | hsa-miR-423-3p | 0.39 | 0.39 | 0.57 |
| 169 | hsa-miR-6083 | — | — | — |
| 170 | hsa-miR-1298-5p | 0.78 | 0.78 | 0.28 |
| 171 | hsa-miR-23b-3p | 0.39 | 0.39 | 0.63 |
| 172 | hsa-miR-339-5p | 0.24 | 0.24 | 0.35 |
| 173 | hsa-miR-1287-3p | — | — | — |
| 174 | hsa-miR-199a-3p | 0.36 | 0.36 | 0.49 |
| 175 | hsa-miR-139-3p | — | — | — |

Based on the information on the abundance of each miRNA contained in a total of 96 specimens measured as described above, miRNAs characteristically present in each cancer type among the same miRNAs, that is, markers of each cancer type were selected.

Whether or not the miRNA is characteristic for each cancer type was determined based on whether or not the ratio between the value confirmed in each cancer type and the value confirmed in healthy subjects is different by twice or more. In other words, when a certain miRNA has a value confirmed in a subject suffering from any cancer type that is twice or more or ½ or less of a value confirmed in a healthy person, the miRNA was determined to be characteristic for a subject suffering from the same cancer type and to be a marker indicating the presence of affection of the same cancer type.

### Results of Marker Selection

The miRNAs selected as markers are shown in Table 3. The numerical values in Table 3 are values obtained by averaging the Ct values related to cDNA of each miRNA in each specimen for each specimen type and further normalizing the average values with an internal standard as described above.

TABLE 3

| | | Ratio to healthy subject | | |
|---|---|---|---|---|
| Sequence | | Breast | Pancreatic | Lung |
| number | Name of miRNA | cancer | cancer | cancer |
| 1 | hsa-miR-3613-5p | 2.77 | 9.69 | 34.14 |
| 2 | hsa-miR-574-3p | 1.71 | 1.71 | 1.30 |
| 3 | hsa-miR-125b-5p | 0.14 | 2.61 | 5.98 |
| 4 | hsa-miR-223-5p | 0.18 | 0.56 | 1.07 |
| 5 | hsa-miR-3613-3p | 0.32 | 0.64 | 0.86 |
| 6 | hsa-miR-205-5p | 0.28 | 0.27 | 0.73 |
| 7 | hsa-miR-941 | 0.17 | 0.44 | 0.69 |
| 8 | hsa-miR-324-3p | 0.49 | 0.53 | 0.66 |
| 9 | hsa-miR-30e-5p | 0.05 | 0.23 | 0.56 |
| 10 | hsa-miR-193a-5p | 0.34 | 0.48 | 0.53 |
| 11 | hsa-miR-106b-5p | 0.13 | 0.13 | 0.42 |
| 12 | hsa-miR-4433a-3p | 0.23 | 0.54 | 0.45 |
| 13 | hsa-miR-29c-3p | 0.17 | 0.29 | 0.36 |
| 14 | hsa-miR-190a-5p | 0.17 | 0.26 | 0.41 |
| 15 | hsa-miR-885-5p | 0.21 | 0.21 | 0.66 |
| 16 | hsa-miR-194-5p | 0.15 | 0.50 | 0.35 |
| 17 | hsa-miR-29a-3p | 0.14 | 0.28 | 0.32 |
| 18 | hsa-miR-142-5p | 0.11 | 0.22 | 0.31 |
| 19 | hsa-miR-483-5p | 0.23 | 0.60 | 0.26 |
| 20 | hsa-miR-142-3p | 0.15 | 0.16 | 0.24 |
| 21 | hsa-miR-122-5p | 0.12 | 0.98 | 0.23 |
| 22 | hsa-miR-34a-5p | 0.07 | 0.54 | 0.25 |
| 23 | hsa-miR-375-3p | 0.05 | 0.05 | 0.28 |

As shown in Table 3, a total of 23 types of cancer markers of each cancer type could be identified. For example, the hsa-miR-190a-5p (SEQ ID NO: 14) has been found to be a marker of a low expression target miRNA indicating that an object suffers from breast cancer, pancreatic cancer or lung cancer when its abundance in the object is ½ or less of its abundance in healthy subjects. Furthermore, the magnitude of the expression level of the hsa-miR-190a-5p is larger in the order of subjects of lung cancer, pancreatic cancer, and breast cancer. Therefore, by comparing the comparison target with the expression level related to a known pancreatic cancer subject, it is possible to identify from which cancer type among breast cancer, pancreatic cancer, and lung cancer the object suffers.

### Example 2. Verification of Marker Performance

The number of specimens was increased as described below, and whether or not the miRNA marker obtained in Example 1 could distinguish from other cancer types was verified.

### Preparation of Specimens

As specimens, a total of 581 specimens of breast cancer patient sera, pancreatic cancer patient sera, lung cancer patient sera, gastric cancer patient sera, colorectal cancer patient sera, and healthy person sera were prepared. As shown in Table 4, the number of specimens was 99 specimens of breast cancer patient sera, 98 specimens of pancreatic cancer patient sera, 98 specimens of lung cancer patient sera, 99 specimens of gastric cancer patient sera, 100 specimens of colorectal cancer patient sera, and 87 specimens of healthy person sera.

TABLE 4

| | Number of specimens |
|---|---|
| Breast cancer | 99 |
| Pancreatic cancer | 98 |
| Lung cancer | 98 |
| Gastric cancer | 99 |
| Colorectal cancer | 100 |
| Healthy subject | 87 |

Treatment of Specimens and Quantification of miRNAs

In the above specimens, miRNAs were extracted in the same manner as in Example 1, and various miRNAs were quantified. Each quantitative value was corrected with the quantitative value of hsa-miR-486-5p as a standard miRNA, and the expression level ratio was determined among the groups of healthy person, breast cancer, lung cancer, pancreatic cancer, gastric cancer, and colorectal cancer.

### Quantitative Results and Statistical Verification

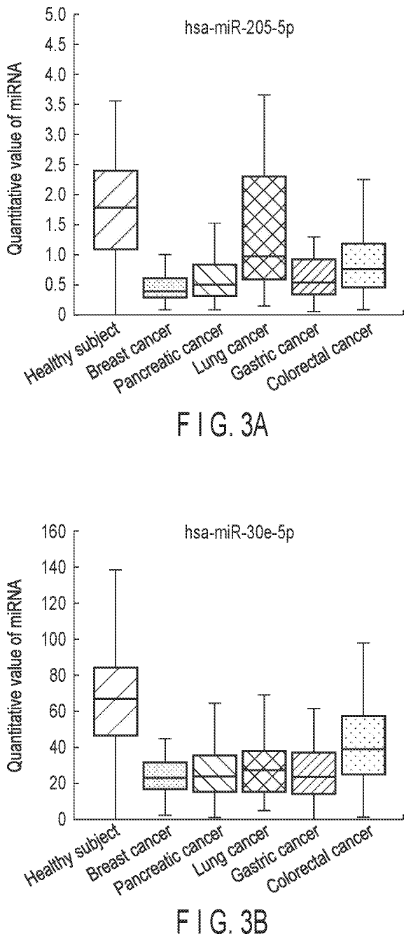

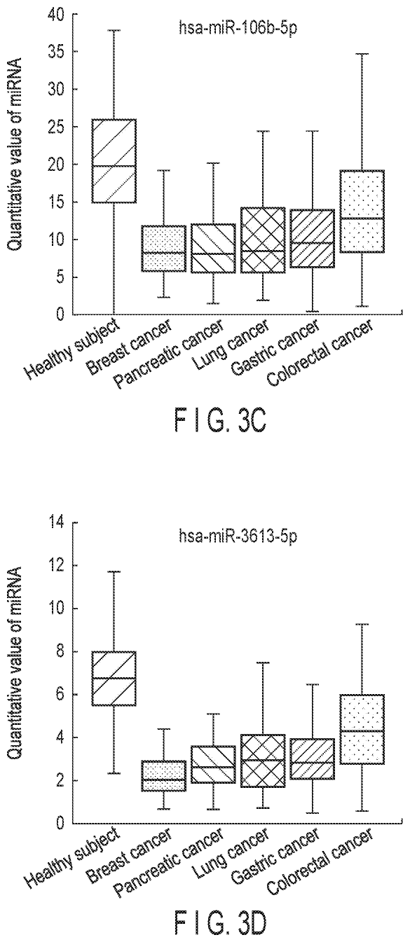

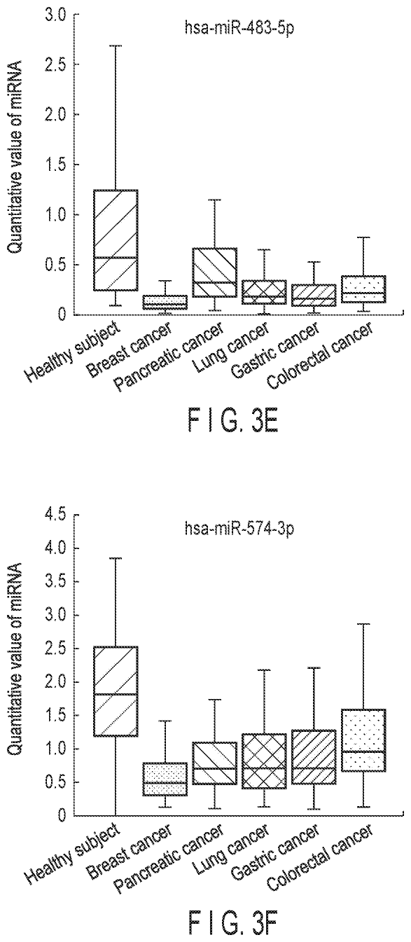

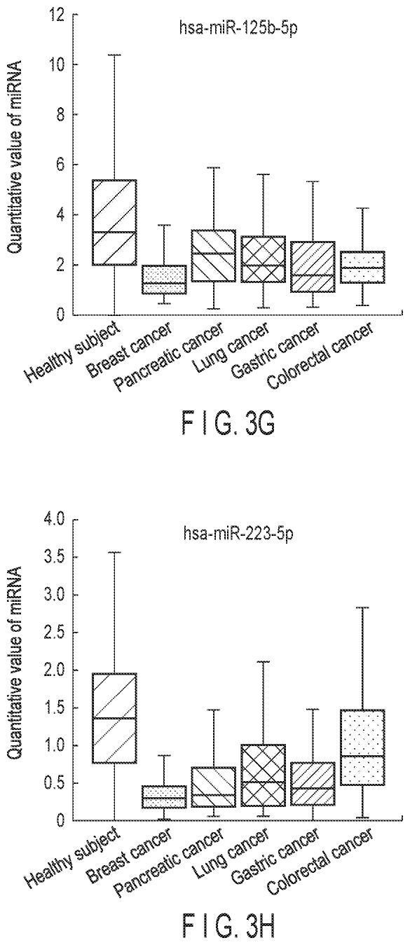

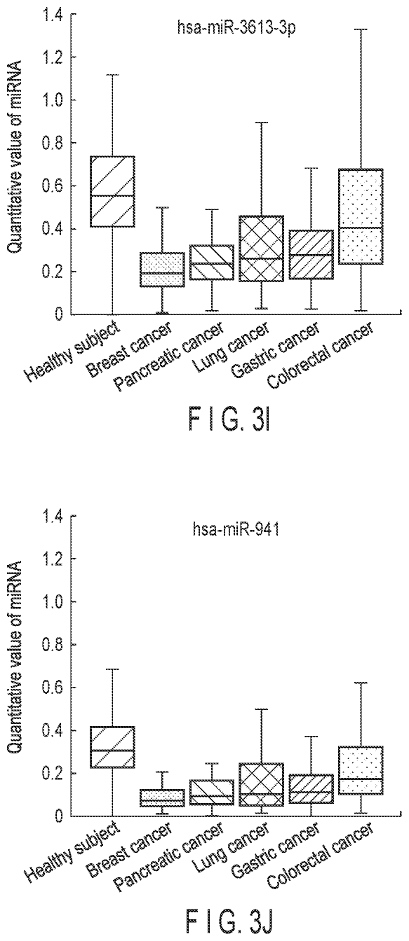

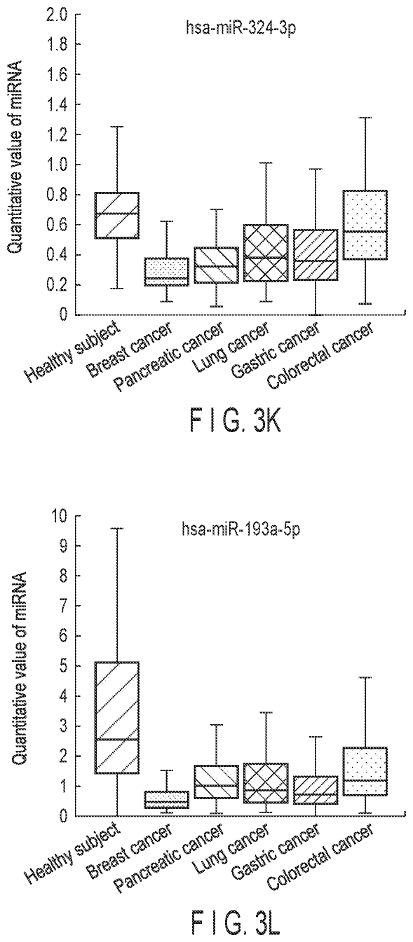

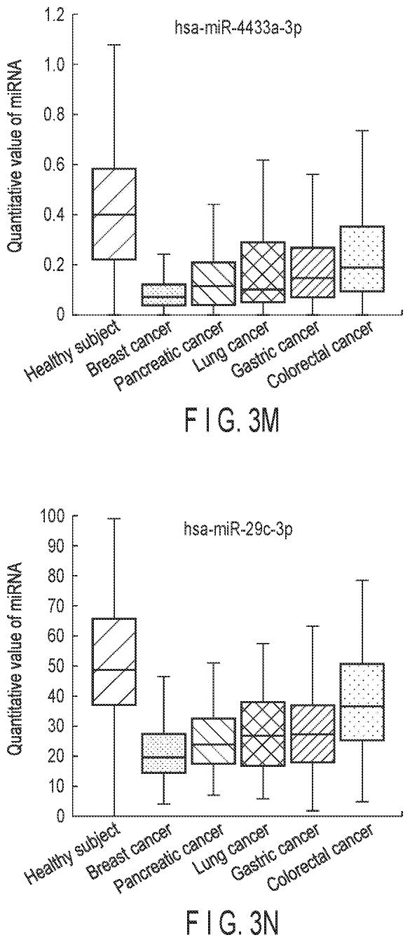

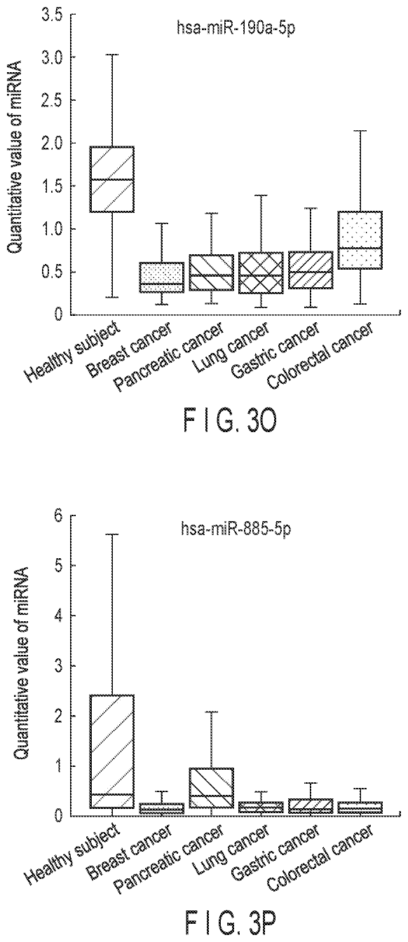

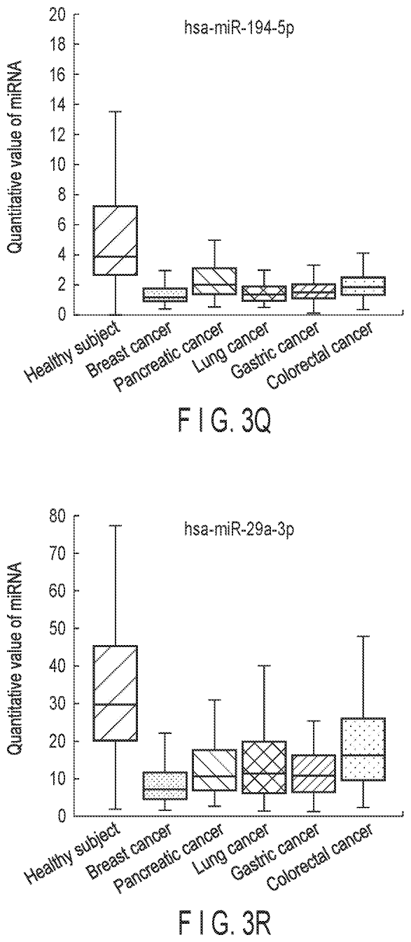

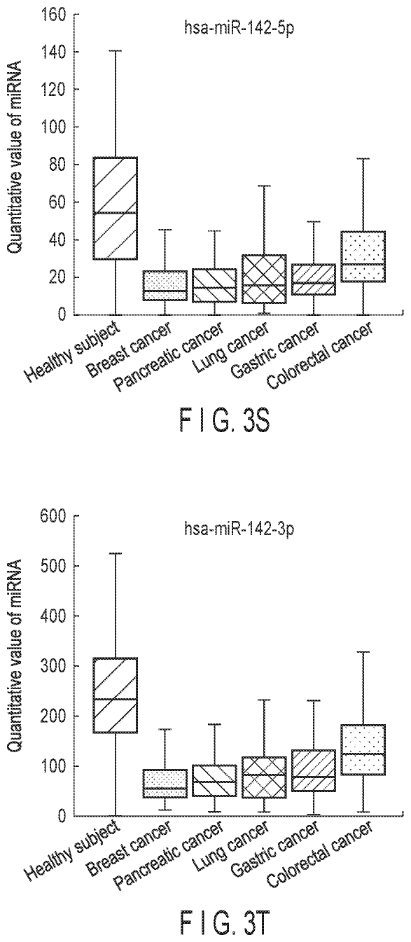

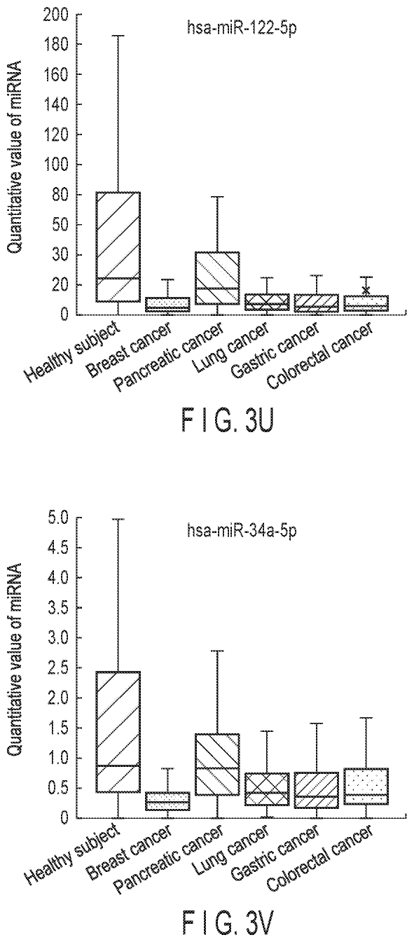

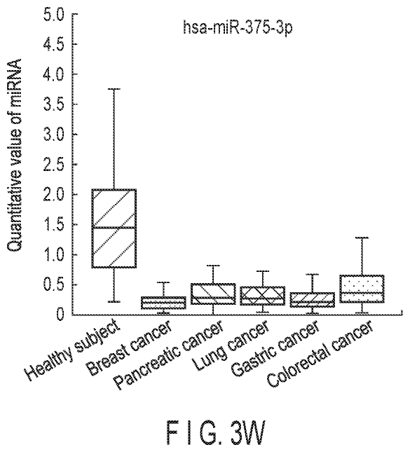

Quantitative values of the 23 miRNA markers identified in Example 2 are shown in FIGS. 3A to 3W. FIGS. 3A to 3W are box plots, in which the horizontal axis represents each cancer type from which each specimen suffers, and the vertical axis represents a relative ratio measured in each specimen.

It was verified whether or not each miRNA marker showing the distribution of quantitative values shown in FIGS. 3A to 3W could distinguish from healthy subjects or other cancer types. Specifically, for example, the control was a healthy subject, and the object was an object suffering from breast cancer, pancreatic cancer, lung cancer, gastric cancer, or colorectal cancer, and it was verified whether or not the possible numerical ranges of the quantitative values of respective miRNAs were statistically different between the two objects. Similarly, it was verified whether or not each marker could statistically distinguish also when the control was a colorectal cancer subject and the object was an object suffering from breast cancer, pancreatic cancer, lung cancer or gastric cancer, when the control was a breast cancer subject and the object was an object suffering from pancreatic cancer, lung cancer or gastric cancer subject, and when the control was a lung cancer subject and the object was pancreatic cancer or gastric cancer.

The results of statistical analysis are shown in Tables 5 to 8. In Tables 5 and 6, “*” indicates significance when the level of significance (p-value) is set to a value smaller than 0.05, “**” indicates significance when the level of significance (p-value) is set to a value smaller than 0.01, “***” indicates significance when the level of significance (p-value) is set to a value smaller than 0.001, and “n.s” indicates no significant difference. In Tables 7 and 8, “decrease” indicates that the quantitative value of the object is smaller than that of the control, “increase” indicates that the quantitative value of the object is larger than that of the control, and “n.s” indicates that it is not a significant difference. Each miRNA is indicated by an abbreviation (for example, the hsa-miR-190a-5p is described as “190a-5p”).

TABLE 5

| | | 205- | 30e- | 106b- | 3613- | 483- | 3613- | 29c- | 190a- | 885- | 223- | | 324- |
|---|---|---|---|---|---|---|---|---|---|---|---|---|---|
| Control | Object | 5p | 5p | 5p | 5p | 5p | 3p | 3p | 5p | 5p | 5p | 941 | 3p |
| Healthy | Breast | *** | *** | *** | *** | *** | *** | *** | *** | *** | *** | *** | *** |
| subject | cancer | | | | | | | | | | | | |
| | Pancreatic | *** | *** | *** | *** | n.s. | *** | *** | *** | *** | *** | *** | *** |
| | cancer | | | | | | | | | | | | |
| | Lung | n.s. | *** | *** | *** | ** | *** | *** | *** | *** | *** | *** | *** |
| | cancer | | | | | | | | | | | | |
| | Gastric | *** | *** | *** | *** | n.s. | *** | *** | *** | *** | *** | *** | *** |
| | cancer | | | | | | | | | | | | |
| | Colorectal | *** | *** | *** | *** | * | n.s. | *** | *** | *** | *** | n.s. | * |
| | cancer | | | | | | | | | | | | |
| Colorectal | Breast | n.s. | *** | *** | *** | n.s. | *** | *** | *** | n.s. | *** | *** | *** |
| cancer | cancer | | | | | | | | | | | | |
| | Pancreatic | n.s. | *** | *** | *** | n.s. | *** | *** | *** | n.s. | *** | *** | *** |
| | cancer | | | | | | | | | | | | |
| | Lung | *** | *** | n.s. | *** | n.s. | *** | *** | *** | n.s. | *** | *** | *** |
| | cancer | | | | | | | | | | | | |
| | Gastric | n.s. | *** | * | *** | n.s. | *** | * | *** | n.s. | *** | *** | *** |
| | cancer | | | | | | | | | | | | |
| Breast | Pancreatic | n.s. | n.s. | n.s. | n.s. | n.s. | n.s. | n.s. | n.s. | n.s. | n.s. | n.s. | n.s. |
| cancer | cancer | | | | | | | | | | | | |
| | Lung | *** | n.s. | n.s. | n.s. | n.s. | * | n.s. | n.s. | n.s. | * | n.s. | * |
| | cancer | | | | | | | | | | | | |
| | Gastric | n.s. | n.s. | n.s. | n.s. | n.s. | n.s. | ** | n.s. | n.s. | n.s. | n.s. | n.s. |
| | cancer | | | | | | | | | | | | |
| Lung | Pancreatic | *** | n.s. | n.s. | n.s. | n.s. | n.s. | n.s. | n.s. | n.s. | n.s. | n.s. | n.s. |
| cancer | cancer | | | | | | | | | | | | |
| | Gastric | *** | n.s. | n.s. | n.s. | n.s. | n.s. | n.s. | n.s. | n.s. | n.s. | n.s. | n.s. |
| | cancer | | | | | | | | | | | | |

*: p < 0.05
**: p < 0.01
***: p < 0.001
n.s: No significant difference

TABLE 6

| | | 142- | 34a- | 375- | 574- | 125b- | 193a- | 4433a- | 194- | 29a- | 142- | 122- |
|---|---|---|---|---|---|---|---|---|---|---|---|---|
| Control | Object | 3p | 5p | 3p | 3p | 5p | 5p | 3p | 5p | 3p | 5p | 5p |
| Healthy | Breast | *** | *** | n.s. | *** | *** | *** | *** | *** | *** | *** | *** |
| subject | cancer | | | | | | | | | | | |
| | Pancreatic | *** | ** | * | *** | * | *** | *** | ** | *** | *** | n.s. |
| | cancer | | | | | | | | | | | |
| | Lung | *** | *** | n.s. | *** | ** | *** | *** | *** | *** | *** | *** |
| | cancer | | | | | | | | | | | |
| | Gastric | *** | *** | * | *** | n.s. | *** | *** | * | *** | *** | n.s. |
| | cancer | | | | | | | | | | | |
| | Colorectal | *** | *** | n.s. | *** | ** | *** | *** | * | *** | *** | *** |
| | cancer | | | | | | | | | | | |
| Colorectal | Breast | *** | n.s. | n.s. | *** | n.s. | *** | *** | n.s. | *** | *** | n.s. |
| cancer | cancer | | | | | | | | | | | |
| | Pancreatic | *** | n.s. | n.s. | ** | n.s. | n.s. | ** | n.s. | * | *** | n.s. |
| | cancer | | | | | | | | | | | |
| | Lung | *** | n.s. | n.s. | ** | n.s. | n.s. | n.s. | n.s. | * | n.s. | n.s. |
| | cancer | | | | | | | | | | | |
| | Gastric | *** | n.s. | n.s. | n.s. | n.s. | n.s. | n.s. | n.s. | n.s. | ** | n.s. |
| | cancer | | | | | | | | | | | |
| Breast | Pancreatic | n.s. | ** | n.s. | n.s. | n.s. | n.s. | n.s. | n.s. | n.s. | n.s. | n.s. |
| cancer | cancer | | | | | | | | | | | |
| | Lung | n.s. | n.s. | n.s. | n.s. | n.s. | n.s. | * | n.s. | n.s. | n.s. | n.s. |
| | cancer | | | | | | | | | | | |
| | Gastric | n.s. | * | n.s. | ** | ** | n.s. | n.s. | n.s. | n.s. | n.s. | * |
| | cancer | | | | | | | | | | | |
| Lung | Pancreatic | n.s. | n.s. | n.s. | n.s. | n.s. | n.s. | n.s. | n.s. | n.s. | n.s. | n.s. |
| cancer | cancer | | | | | | | | | | | |
| | Gastric | n.s. | n.s. | n.s. | n.s. | n.s. | n.s. | n.s. | n.s. | n.s. | n.s. | * |
| | cancer | | | | | | | | | | | |

*: p < 0.05
**: p < 0.01
***: p < 0.001
n.s: No significant difference

TABLE 7

| Con- | Ob- | 205- | 30e- | 106b- | 3613- | 483- | 3613- |
|---|---|---|---|---|---|---|---|
| trol | ject | 5p | 5p | 5p | 5p | 5p | 3p |
| Healthy | Breast | Decrease | Decrease | Decrease | Decrease | Decrease | Decrease |
| subject | cancer | | | | | | |
| | Pancreatic | Decrease | Decrease | Decrease | Decrease | n.s. | Decrease |
| | cancer | | | | | | |
| | Lung | n.s. | Decrease | Decrease | Decrease | Decrease | Decrease |
| | cancer | | | | | | |
| | Gastric | Decrease | Decrease | Decrease | Decrease | n.s. | Decrease |
| | cancer | | | | | | |
| | Colorectal | Decrease | Decrease | Decrease | Decrease | Decrease | n.s. |
| | cancer | | | | | | |
| Colorectal | Breast | n.s. | Decrease | Decrease | Decrease | n.s. | Decrease |
| cancer | cancer | | | | | | |
| | Pancreatic | n.s. | Decrease | Decrease | Decrease | n.s. | Decrease |
| | cancer | | | | | | |
| | Lung | Increase | Decrease | n.s. | Decrease | n.s. | Decrease |
| | cancer | | | | | | |
| | Gastric | n.s. | Decrease | Decrease | Decrease | n.s. | Decrease |
| | cancer | | | | | | |
| Breast | Pancreatic | n.s. | n.s. | n.s. | n.s. | n.s. | n.s. |
| cancer | cancer | | | | | | |
| | Lung | Increase | n.s. | n.s. | n.s. | n.s. | Increase |
| | cancer | | | | | | |
| | Gastric | n.s. | n.s. | n.s. | n.s. | n.s. | n.s. |
| | cancer | | | | | | |
| Lung | Pancreatic | Decrease | n.s. | n.s. | n.s. | n.s. | n.s. |
| cancer | cancer | | | | | | |
| | Gastric | Decrease | n.s. | n.s. | n.s. | n.s. | n.s. |
| | cancer | | | | | | |
| Con- | Ob- | 29c- | 190a- | 885- | 223- | | 324- |
| trol | ject | 3p | 5p | 5p | 5p | 941 | 3p |
| Healthy | Breast | Decrease | Decrease | Decrease | Decrease | Decrease | Decrease |
| subject | cancer | | | | | | |
| | Pancreatic | Decrease | Decrease | Decrease | Decrease | Decrease | Decrease |
| | cancer | | | | | | |
| | Lung | Decrease | Decrease | Decrease | Decrease | Decrease | Decrease |
| | cancer | | | | | | |
| | Gastric | Decrease | Decrease | Decrease | Decrease | Decrease | Decrease |
| | cancer | | | | | | |
| | Colorectal | Decrease | Decrease | Decrease | Decrease | n.s. | Decrease |
| | cancer | | | | | | |
| Colorectal | Breast | Decrease | Decrease | n.s. | Decrease | Decrease | Decrease |
| cancer | cancer | | | | | | |
| | Pancreatic | Decrease | Decrease | n.s. | Decrease | Decrease | Decrease |
| | cancer | | | | | | |
| | Lung | Decrease | Decrease | n.s. | Decrease | Decrease | Decrease |
| | cancer | | | | | | |
| | Gastric | Decrease | Decrease | n.s. | Decrease | Decrease | Decrease |
| | cancer | | | | | | |
| Breast | Pancreatic | n.s. | n.s. | n.s. | n.s. | n.s. | n.s. |
| cancer | cancer | | | | | | |
| | Lung | n.s. | n.s. | n.s. | Increase | n.s. | Increase |
| | cancer | | | | | | |
| | Gastric | Increase | n.s. | n.s. | n.s. | n.s. | n.s. |
| | cancer | | | | | | |
| Lung | Pancreatic | n.s. | n.s. | n.s. | n.s. | n.s. | n.s. |
| cancer | cancer | | | | | | |
| | Gastric | n.s. | n.s. | n.s. | n.s. | n.s. | n.s. |
| | cancer | | | | | | |

Decrease: Quantitative value of object is smaller than that of control
Increase: Quantitative value of object is larger than that of control
n.s: No significant difference

TABLE 8

| | | 142- | 34a- | 375- | 574- | 125b- | 193a- | 4433a- | 194- | 29a- | 142- | 122- |
|---|---|---|---|---|---|---|---|---|---|---|---|---|
| Control | Object | 3p | 5p | 3p | 3p | 5p | 5p | 3p | 5p | 3p | 5p | 5p |
| Healthy | Breast | Decrease | Decrease | n.s. | Decrease | Decrease | Decrease | Decrease | Decrease | Decrease | Decrease | Decrease |
| subject | cancer | | | | | | | | | | | |
| | Pancre- | Decrease | Decrease | Decrease | Decrease | Decrease | Decrease | Decrease | Decrease | Decrease | Decrease | n.s. |
| | atic | | | | | | | | | | | |
| | cancer | | | | | | | | | | | |
| | Lung | Decrease | Decrease | n.s. | Decrease | Decrease | Decrease | Decrease | Decrease | Decrease | Decrease | Decrease |
| | cancer | | | | | | | | | | | |
| | Gastric | Decrease | Decrease | Decrease | Decrease | n.s. | Decrease | Decrease | Decrease | Decrease | Decrease | n.s. |
| | cancer | | | | | | | | | | | |
| | Colo- | Decrease | Decrease | n.s. | Decrease | Decrease | Decrease | Decrease | Decrease | Decrease | Decrease | Decrease |
| | rectal | | | | | | | | | | | |
| | cancer | | | | | | | | | | | |
| Colo- | Breast | Decrease | n.s. | n.s. | Decrease | n.s. | Decrease | Decrease | n.s. | Decrease | Decrease | n.s. |
| rectal | cancer | | | | | | | | | | | |
| cancer | Pancre- | Decrease | n.s. | n.s. | Decrease | n.s. | n.s. | Decrease | n.s. | Decrease | Decrease | n.s. |
| | atic | | | | | | | | | | | |
| | cancer | | | | | | | | | | | |
| | Lung | Decrease | n.s. | n.s. | Decrease | n.s. | n.s. | n.s. | n.s. | Decrease | n.s. | n.s. |
| | cancer | | | | | | | | | | | |
| | Gastric | Decrease | n.s. | n.s. | n.s. | n.s. | n.s. | n.s. | n.s. | n.s. | Decrease | n.s. |
| | cancer | | | | | | | | | | | |
| Breast | Pancre- | n.s. | Increase | n.s. | n.s. | n.s. | n.s. | n.s. | n.s. | n.s. | n.s. | n.s. |
| cancer | atic | | | | | | | | | | | |
| | cancer | | | | | | | | | | | |
| | Lung | n.s. | n.s. | n.s. | n.s. | n.s. | n.s. | Increase | n.s. | n.s. | n.s. | n.s. |
| | cancer | | | | | | | | | | | |
| | Gastric | n.s. | Increase | n.s. | Increase | Increase | n.s. | n.s. | n.s. | n.s. | n.s. | Increase |
| | cancer | | | | | | | | | | | |
| Lung | Pancre- | n.s. | n.s. | n.s. | n.s. | n.s. | n.s. | n.s. | n.s. | n.s. | n.s. | n.s. |
| cancer | atic | | | | | | | | | | | |
| | cancer | | | | | | | | | | | |
| | Gastric | n.s. | n.s. | n.s. | n.s. | n.s. | n.s. | n.s. | n.s. | n.s. | n.s. | Increase |
| | cancer | | | | | | | | | | | |

Decrease: Quantitative value of object is smaller than that of control
Increase: Quantitative value of object is larger than that of control
n.s: No significant difference

For example, the relative ratio of hsa-miR-30e-5p was significantly lower in all cancers than that in healthy subjects, and was significantly reduced in breast, pancreas, lung, and gastric cancer as compared to that in colorectal cancer among cancer specimens (healthy subject vs breast, pancreas, lung, gastric or colorectal cancer, p<0.001; colorectal cancer vs breast, pancreas, lung or gastric cancer, p<0.001). The relative ratio of hsa-miR-3613-5p was significantly lower in all cancers than that in healthy subjects, and was significantly reduced in breast cancer, pancreatic cancer, lung cancer, and gastric cancer as compared to that in colorectal cancer among cancer specimens (healthy subject vs breast, pancreas, lung, gastric or colorectal cancer, p<0.001; colorectal cancer vs breast, pancreas, lung or gastric cancer, p<0.001). The relative ratio of hsa-miR-190a-5p was significantly lower in all cancer types than that in healthy subjects, and was significantly reduced in breast cancer, pancreatic cancer, lung cancer, and gastric cancer as compared to that in colorectal cancer among cancer specimens (healthy subject vs breast, pancreas, lung, gastric or colorectal cancer, p<0.001; colorectal cancer vs breast, pancreas, lung or gastric cancer, p<0.001). The relative ratio of hsa-miR-194-5p was significantly reduced in all cancers as compared to that in healthy subjects, but no significant difference was observed between cancer specimens (healthy subject vs breast or lung cancer, p<0.001; healthy subject vs pancreatic cancer, p<0.01; healthy subject vs gastric or colorectal cancer, p<0.05). The relative ratio of hsa-miR-223-5p was significantly lower in all cancers than that in healthy subjects, was significantly reduced in breast cancer, pancreatic cancer, lung cancer, gastric cancer as compared to that in colorectal cancer among cancer specimens, and breast cancer was significantly increased as compared to lung cancer (healthy subject vs breast, pancreas, lung, gastric or colorectal cancer, p<0.001; colorectal cancer vs breast, pancreas, lung, or gastric cancer, p<0.001; breast cancer vs lung cancer, p<0.05). The relative ratio of hsa-miR-3613-3p was significantly lower in breast, pancreatic, lung, and gastric cancer than that in healthy subjects, was significantly reduced in breast, pancreatic, lung, and gastric cancer as compared to that in colorectal cancer among cancer specimens, and was significantly increased in lung cancer as compared to that in breast cancer (healthy subject vs breast, pancreas, lung or gastric cancer, p<0.001; colorectal cancer vs breast, pancreas, lung or gastric cancer p<0.001; breast cancer vs lung cancer, p<0.05).

### Example 3. Extraction and Verification of Combination of miRNAs Identifying Various Cancer Subjects

### Extraction of Combination

Using randomly extracted 80% of all the data obtained in Example 2 as learning data, a combination of markers for identifying colorectal cancer or pancreatic cancer among the 23 microRNAs shown in Table 1 was extracted by the degree of contribution of a binary logistic regression model or a multinomial logistic regression model.

### Results

As a result of selecting combinations of miRNAs, it has been found that combinations 1 to 6 shown in Table 9 and Table 10 are statistically significant.

TABLE 9

| | Sequence | Name of | Base |
|---|---|---|---|
| | number | miRNA | sequence |
| Combination 1 | 4 | hsa-miR-223-5p | CGUGUAU |
| | | | UUGACAA |
| | | | GCUGAGU |
| | | | U |
| | 6 | hsa-miR-205-5p | UCCUUCA |
| | | | UUCCACC |
| | | | GGAGUCU |
| | | | G |
| | 9 | hsa-miR-30e-5p | UGUAAAC |
| | | | AUCCUUG |
| | | | ACUGGAA |
| | | | G |
| | 11 | hsa-miR-106b-5p | UAAAGUG |
| | | | CUGACAG |
| | | | UGCAGAU |
| | 13 | hsa-miR-29c-3p | UAGCACC |
| | | | AUUUGAA |
| | | | AUCGGUU |
| | | | A |
| | 176 | hsa-miR-486-5p | UCCUGUA |
| | | | CUGAGCU |
| | | | GCCCCGA |
| | | | G |
| Combination 2 | 6 | hsa-miR-205-5p | UCCUUCA |
| | | | UUCCACC |
| | | | GGAGUCU |
| | | | G |
| | 8 | hsa-miR-324-3p | CCCACUG |
| | | | CCCCAGG |
| | | | UGCUGCU |
| | | | GG |
| | 9 | hsa-miR-30e-5p | UGUAAAC |
| | | | AUCCUUG |
| | | | ACUGGAA |
| | | | G |
| | 11 | hsa-miR-106b-5p | UAAAGUG |
| | | | CUGACAG |
| | | | UGCAGAU |
| | 15 | hsa-miR-885-5p | UCCAUUA |
| | | | CACUACC |
| | | | CUGCCUC |
| | | | U |
| | 176 | hsa-miR-486-5p | UCCUGUA |
| | | | CUGAGCU |
| | | | GCCCCGA |
| | | | G |
| Combination 3 | 6 | hsa-miR-205-5p | UCCUUCA |
| | | | UUCCACC |
| | | | GGAGUCU |
| | | | G |
| | 9 | hsa-miR-30e-5p | UGUAAAC |
| | | | AUCCUUG |
| | | | ACUGGAA |
| | | | G |
| | 11 | hsa-miR-106b-5p | UAAAGUG |
| | | | CUGACAG |
| | | | UGCAGAU |
| | 13 | hsa-miR-29c-3p | UAGCACC |
| | | | AUUUGAA |
| | | | AUCGGUU |
| | | | A |
| | 15 | hsa-miR-885-5p | UCCAUUA |
| | | | CACUACC |
| | | | CUGCCUC |
| | | | U |
| | 176 | hsa-miR-486-5p | UCCUGUA |
| | | | CUGAGCU |
| | | | GCCCCGA |
| | | | G |

TABLE 10

| | Sequence | | Base |
|---|---|---|---|
| | number | Name of miRNA | sequence |
| Combination 4 | 1 | hsa-miR-3613-5p | UGUUGUA |
| | | | CUUUUUU |
| | | | UUUUGUU |
| | | | C |
| | 5 | hsa-miR-3613-3p | ACAAAAA |
| | | | AAAAAGC |
| | | | CCAACCC |
| | | | UUC |
| | 6 | hsa-miR-205-5p | UCCUUCA |
| | | | UUCCACC |
| | | | GGAGUCU |
| | | | G |
| | 14 | hsa-miR-190a-5p | UGAUAUG |
| | | | UUUGAUA |
| | | | UAUUAGG |
| | | | U |
| | 19 | hsa-miR-483-5p | AAGACGG |
| | | | GAGGAAA |
| | | | GAAGGGA |
| | | | G |
| | 176 | hsa-miR-486-5p | UCCUGUA |
| | | | CUGAGCU |
| | | | GCCCCGA |
| | | | G |
| Combination 5 | 1 | hsa-miR-3613-5p | UGUUGUA |
| | | | CUUUUUU |
| | | | UUUUGUU |
| | | | C |
| | 5 | hsa-miR-3613-3p | ACAAAAA |
| | | | AAAAAGC |
| | | | CCAACCC |
| | | | UUC |
| | 7 | hsa-miR-941 | CACCCGG |
| | | | CUGUGUG |
| | | | CACAUGU |
| | | | GC |
| | 19 | hsa-miR-483-5p | AAGACGG |
| | | | GAGGAAA |
| | | | GAAGGGA |
| | | | G |
| | 23 | hsa-miR-375-3p | UUUGUUC |
| | | | GUUCGGC |
| | | | UCGCGUG |
| | | | A |
| | 176 | hsa-miR-486-5p | UCCUGUA |
| | | | CUGAGCU |
| | | | GCCCCGA |
| | | | G |
| Combination 6 | 1 | hsa-miR-3613-5p | UGUUGUA |
| | | | CUUUUUU |
| | | | UUUUGUU |
| | | | C |
| | 14 | hsa-miR-190a-5p | UGAUAUG |
| | | | UUUGAUA |
| | | | UAUUAGG |
| | | | U |
| | 18 | hsa-miR-142-3p | UGUAGUG |
| | | | UUUCCUA |
| | | | CUUUAUG |
| | | | GA |
| | 19 | hsa-miR-483-5p | AAGACGG |
| | | | GAGGAAA |
| | | | GAAGGGA |
| | | | G |
| | 22 | hsa-miR-34a-5p | UGGCAGU |
| | | | GUCUUAG |
| | | | CUGGUUG |
| | | | U |
| | 176 | hsa-miR-486-5p | UCCUGUA |
| | | | CUGAGCU |
| | | | GCCCCGA |
| | | | G |

Verification of Identification Performance by Combination of miRNAs

With respect to the combination of miRNAs obtained by the extraction, identification performance was verified by a discriminant model using data other than learning data as test data.

### Verification Result

Table 11 and Table 12 show the identification performance of each combination of miRNAs.

TABLE 11

| | Sensitivity | Specificity |
|---|---|---|
| | (%) | (%) |
| Combination 1 | 80 | 51 |
| Combination 2 | 80 | 58 |
| Combination 3 | 80 | 56 |

TABLE 12

| | Sensitivity | Specificity |
|---|---|---|
| | (%) | (%) |
| Combination 4 | 80 | 58 |
| Combination 5 | 80 | 53 |
| Combination 6 | 80 | 65 |

As shown in Table 11, it has been found that, for example, affection of colorectal cancer can be discriminated with a sensitivity of 80% by obtaining a quantitative value of combination 1 from test data including a healthy subject, a breast cancer-affected specimen, a pancreatic cancer-affected specimen, a lung cancer-affected specimen, a gastric cancer-affected specimen, and a colorectal cancer-affected specimen, and making determination using a colorectal cancer discriminant model obtained from learning data including a healthy subject, a breast cancer-affected specimen, a pancreatic cancer-affected specimen, a lung cancer-affected specimen, a gastric cancer-affected specimen, and a colorectal cancer-affected specimen. Therefore, the presence of affection of a colorectal cancer of the object can be confirmed by obtaining the quantitative value of combination 1 of miRNAs in the object.

Similarly, the presence of affection of colorectal cancer can be also confirmed by obtaining quantitative values of combinations 2 and 3 from test data including non-cancer, colorectal cancer, breast cancer, pancreatic cancer, lung cancer, and gastric cancer-affected specimens, and making determination using a colorectal cancer discriminant model obtained from learning data including non-cancer, colorectal cancer, breast cancer, pancreatic cancer, lung cancer, and gastric cancer-affected specimens (see Table 11).

In addition, as shown in Table 12, it has been found that affection of pancreatic cancer can be discriminated with a sensitivity of 80% by obtaining quantitative values of combinations 4 to 6 from test data including a healthy subject, a breast cancer-affected specimen, a pancreatic cancer-affected specimen, a lung cancer-affected specimen, a gastric cancer-affected specimen, and a colorectal cancer-affected specimen, and making determination using a pancreatic cancer discriminant model obtained from learning data including a healthy subject, a breast cancer-affected specimen, a pancreatic cancer-affected specimen, a lung cancer-affected specimen, a gastric cancer-affected specimen, and a colorectal cancer-affected specimen. Therefore, the presence of affection of pancreatic cancer can be confirmed by obtaining the quantitative values of combinations 4 to 6 of miRNAs in the object.

As described above, according to Example 3, it was shown that combinations 1 to 6 of miRNAs presented in Example 2 are excellent as markers for identifying a cancer type.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An analysis method for determining a presence of an affection of at least any one of breast cancer, pancreatic cancer, lung cancer, gastric cancer, and colorectal cancer in an object, the method comprising:
quantifying miRNA selected from a miRNA group consisting of hsa-miR-205-5p, hsa-miR-30e-5p, hsa-miR-106b-5p, hsa-miR-3613-5p, hsa-miR-483-5p, hsa-miR-574-3p, hsa-miR-125b-5p, hsa-miR-223-5p, hsa-miR-3613-3p, hsa-miR-941, hsa-miR-324-3p, hsa-miR-193a-5p, hsa-miR-4433a-3p, hsa-miR-29c-3p, hsa-miR-190a-5p, hsa-miR-885-5p, hsa-miR-194-5p, hsa-miR-29a-3p, hsa-miR-142-5p, hsa-miR-142-3p, hsa-miR-122-5p, hsa-miR-34a-5p, and hsa-miR-375-3p in a first sample, derived from the object, to obtain a first quantitative miRNA value.

2. The method of claim 1, further comprising:
quantifying the miRNA in a second sample, derived from a control, to obtain a second quantitative miRNA value; and
comparing the first quantitative miRNA value in the object with the second quantitative miRNA value in the control to determine that the object suffers from at least any one of breast cancer, pancreatic cancer, lung cancer, gastric cancer, and colorectal cancer.

3. The method of claim 2, wherein the control is a healthy subject or a specimen known to be at least any one of breast cancer, pancreatic cancer, lung cancer, gastric cancer, and colorectal cancer.

4. The method of claim 3, wherein
the control is a healthy subject, and
wherein the determining determines that the object suffers from breast cancer, pancreatic cancer, lung cancer, gastric cancer or colorectal cancer when the first quantitative miRNA value of any one miRNA selected from the group consisting of hsa-miR-30e-5p, hsa-miR-106b-5p, hsa-miR-3613-5p, hsa-miR-574-3p, hsa-miR-223-5p, hsa-miR-324-3p, hsa-miR-193a-5p, hsa-miR-4433a-3p, hsa-miR-29c-3p, hsa-miR-190a-5p, hsa-miR-194-5p, hsa-miR-29a-3p, hsa-miR-142-5p, hsa-miR-142-3p, hsa-miR-34a-5p, and hsa-miR-885-5p in the object is smaller than the second quantitative miRNA value in the control.

5. The method of claim 3, wherein the control is a healthy subject, and
wherein the determining determines that the object suffers from breast cancer, pancreatic cancer, lung cancer, or colorectal cancer when the first quantitative miRNA value of hsa-miR-125b-5p in the object is smaller than the second quantitative miRNA value in the control.

6. The method of claim 3, wherein the control is a healthy subject, and
wherein the determining determines that the object suffers from breast cancer, pancreatic cancer, lung cancer, or gastric cancer when the first quantitative miRNA value of hsa-miR-3613-3p or hsa-miR-941 in the object is smaller than the second quantitative miRNA value in the control.

7. The method of claim 3, wherein the control is a healthy subject, and
wherein the determining determines that the object suffers from breast cancer, pancreatic cancer, gastric cancer, or colorectal cancer when the first quantitative miRNA value of hsa-miR-205-5p in the object is smaller than the second quantitative miRNA value in the control.

8. The method of claim 3, wherein the control is a healthy subject, and
wherein the determining determines that the object suffers from breast cancer, lung cancer, or colorectal cancer when first the miRNA quantitative value of hsa-miR-483-5p or hsa-miR-122-5p in the object is smaller than second the miRNA quantitative value in the control.

9. The method of claim 3, wherein the control is a healthy subject, and
wherein the determining determines that the object suffers from pancreatic cancer or gastric cancer when the first quantitative miRNA value of hsa-miR-375-3p in the object is smaller than the second quantitative miRNA value in the control.

10. The method of claim 3, wherein the control is a colorectal cancer subject, and
wherein the determining determines that the object suffers from breast cancer, pancreatic cancer, lung cancer, or gastric cancer when the first quantitative miRNA value of hsa-miR-30c-5p, hsa-miR-3613-5p, hsa-miR-223-5p, hsa-miR-3613-3p, hsa-miR-941, hsa-miR-324-3p, hsa-miR-29c-3p, hsa-miR-190a-5p, or hsa-miR-142-3p in the object is smaller than the second quantitative miRNA value in the control.

11. The method of claim 3, wherein the control is a colorectal cancer subject, and
wherein the determining determines that the object suffers from breast cancer, pancreatic cancer, or lung cancer when the first quantitative miRNA value of hsa-miR-574-3p or hsa-miR-29a-3p in the object is smaller than the second quantitative miRNA value in the control.

12. The method of claim 3, wherein the control is a colorectal cancer subject, and
wherein the determining determines that the object suffers from lung cancer when the first quantitative miRNA value of hsa-miR-205-5p in the object is larger than the second quantitative miRNA value in the control.

13. The method of claim 3, wherein the control is a colorectal cancer subject, and
wherein the determining determines that the object suffers from breast cancer, pancreatic cancer, or gastric cancer when the first quantitative miRNA value of hsa-miR-106b-5 or hsa-miR-142-5p in the object is smaller than the second quantitative miRNA value in the control.

14. The method of claim 3, wherein the control is a colorectal cancer subject, and
wherein the determining determines that the object suffers from breast cancer when the first quantitative miRNA value of hsa-miR-193a-5p in the object is smaller than the second quantitative miRNA value in the control.

15. The method of claim 3, wherein the control is a colorectal cancer subject, and
wherein the determining determines that the object suffers from breast cancer or pancreatic cancer when the first quantitative miRNA value of hsa-miR-4433a-3p in the object is smaller than the second quantitative miRNA value in the control.

16. The method of claim 3, wherein the control is a breast cancer subject, and
wherein the determining determines that the object suffers from gastric cancer when the first quantitative miRNA value of hsa-miR-29c-3p, hsa-miR-574-3p, hsa-miR-125b-5p, or hsa-miR-122-5p in the object is larger than the second quantitative miRNA value in the control.

17. The method of claim 3, wherein the control is a breast cancer subject, and
wherein the determining determines that the object suffers from lung cancer when the first quantitative miRNA value of hsa-miR-205-5p, hsa-miR-3613-3p, hsa-miR-223-5p, hsa-miR-324-3p, or hsa-miR-4433a-3p in the object is larger than the second quantitative miRNA value in the control.

18. The method of claim 3, wherein the control is a breast cancer subject, and
wherein the determining determines that the object suffers from pancreatic cancer or gastric cancer when the first quantitative miRNA value of hsa-miR-34a-5p in the object is larger than second the miRNA quantitative value in the control.

19. The method of claim 3, wherein the control is a lung cancer subject, and
wherein the determining determines that the object suffers from pancreatic cancer or gastric cancer when the first quantitative miRNA value of hsa-miR-205-5p in the object is smaller than the second quantitative miRNA value in the control.

20. The method of claim 3, wherein the control is a lung cancer subject, and
wherein the determining determines that the object suffers from gastric cancer when the first quantitative miRNA value of hsa-miR-122-5p in the object is larger than the second quantitative miRNA value in the control.

21. An analysis method for determining a presence of an affection of at least any one of breast cancer, pancreatic cancer, lung cancer, gastric cancer, and colorectal cancer in an object, the method comprising:
quantifying any plurality of miRNAs selected from the miRNA group consisting of hsa-miR-205-5p, hsa-miR-30c-5p, hsa-miR-106b-5p, hsa-miR-3613-5p, hsa-miR-483-5p, hsa-miR-574-3p, hsa-miR-125b-5p, hsa-miR-223-5p, hsa-miR-3613-3p, hsa-miR-941, hsa-miR-324-3p, hsa-miR-193a-5p, hsa-miR-4433a-3p, hsa-miR-29c-3p, hsa-miR-190a-5p, hsa-miR-885-5p, hsa-miR-194-5p, hsa-miR-29a-3p, hsa-miR-142-5p, hsa-miR-142-3p, hsa-miR-122-5p, hsa-miR-34a-5p, and hsa-miR-375-3p in a sample derived from the object, to obtain first quantitative miRNA values.

22. The method of claim 21, further comprising:
quantifying the plurality of miRNAs in a sample derived from a control, to obtain second quantitative miRNA values; and
comparing the first quantitative miRNA values of the plurality of types of miRNAs in the object with the second quantitative miRNA values of the plurality of miRNAs in the control to determine that the object suffers from at least any one of breast cancer, pancreatic cancer, lung cancer, gastric cancer, and colorectal cancer.

23. The method of claim 22, wherein the control is a healthy subject or a specimen known to be at least any one of breast cancer, pancreatic cancer, lung cancer, gastric cancer, and colorectal cancer.

24. The method of claim 21, comprising:
preparing a learning sample derived from a subject other than the object;
quantifying each miRNA of a second miRNA group in the learning sample;
constructing a determination algorithm for determining the presence of affection of at least any one of breast cancer, pancreatic cancer, lung cancer, gastric cancer, and colorectal cancer, using information on the subject and a quantitative value of the second miRNA group in the learning sample as model learning data; and
determining the presence of affection of at least one cancer of breast cancer, pancreatic cancer, lung cancer, gastric cancer, and colorectal cancer in the object from quantitative values of any plurality of miRNAs selected from the miRNA group of the object and the information on the subject by using the determination algorithm.

25. The method of claim 24, wherein the information on the subject is information about cancer affection of the subject.

26. The method of claim 24, wherein the determination algorithm uses a variable selection method using binary logistic regression or multinomial logistic regression, a variable selection method using a statistical method, a variable selection method using a contribution degree and a p value, or a machine learning method.

27. The method of claim 26, wherein the variable selection method using binary logistic regression or multinomial logistic regression is sparse estimation, a stepwise method, or a brute force method, and
wherein the machine learning method is random forest, boosting, or neural network.

28. The method of claim 24, wherein the determining determines the presence of affection of colorectal cancer of the object from quantitative values of hsa-miR-223-5p, hsa-miR-205-5p, hsa-miR-30e-5p, hsa-miR-106b-5p, hsa-miR-29c-3p and hsa-miR-486-5p in the object by using the determination algorithm.

29. The method of claim 24, wherein the determining determines the presence of affection of colorectal cancer of the object from quantitative values of hsa-miR-205-5p, hsa-miR-324-3p, hsa-miR-30e-5p, hsa-miR-106b-5p, hsa-miR-885-5p and hsa-miR-486-5p in the object by using the determination algorithm.

30. The method of claim 24, wherein the determining determines the presence of affection of colorectal cancer of the object from quantitative values of hsa-miR-205-5p, hsa-miR-30e-5p, hsa-miR-106b-5p, hsa-miR-29c-3p, hsa-miR-885-5p and hsa-miR-486-5p in the object by using the determination algorithm.

31. The method of claim 24, wherein the determining determines the presence of affection of pancreatic cancer of the object from quantitative values of hsa-miR-3613-5p, hsa-miR-3613-3p, hsa-miR-205-5p, hsa-miR-190a-5p, hsa-miR-483-5p and hsa-miR-486-5p in the object by using the determination algorithm.

32. The method of claim 24, wherein the determining determines the presence of affection of pancreatic cancer of the object from quantitative values of hsa-miR-3613-5p, hsa-miR-3613-3p, hsa-miR-941, hsa-miR-483-5p, hsa-miR-375-3p and hsa-miR-486-5p in the object by using the determination algorithm.

33. The method of claim 24, wherein the determining determines the presence of affection of pancreatic cancer of the object from quantitative values of hsa-miR-3613-5p, hsa-miR-190a-5p, hsa-miR-142-3p, hsa-miR-483-5p, hsa-miR-34a-5p and hsa-miR-486-5p in the object by using the determination algorithm.

34. The method of claim 1, wherein the quantifying is performed using a PCR method, a LAMP method, or a microarray method.

35. The method of claim 1, wherein the sample is serum or plasma.

36. The method of claim 2, wherein the first quantitative miRNA value of the sample derived from the object and the second quantitative miRNA value of the sample derived from the control are corrected by standard miRNA commonly contained in the sample derived from the object and the sample derived from the control.

37. The method of claim 36, wherein the standard miRNA is hsa-miR-486-5p.

38. A kit configured for detecting at least one of breast cancer, pancreatic cancer, lung cancer, gastric cancer, and colorectal cancer, the kit comprising:
a nucleic acid capable of specifically binding to at least one miRNA selected from the group consisting of hsa-miR-205-5p, hsa-miR-30e-5p, hsa-miR-106b-5p, hsa-miR-3613-5p, hsa-miR-483-5p, hsa-miR-574-3p, hsa-miR-125b-5p, hsa-miR-223-5p, hsa-miR-3613-3p, hsa-miR-941, hsa-miR-324-3p, hsa-miR-193a-5p, hsa-miR-4433a-3p, hsa-miR-29c-3p, hsa-miR-190a-5p, hsa-miR-885-5p, hsa-miR-194-5p, hsa-miR-29a-3p, hsa-miR-142-5p, hsa-miR-142-3p, hsa-miR-122-5p, hsa-miR-34a-5p, and hsa-miR-375-3p.

39. The kit of claim 38, wherein the nucleic acid capable of specifically binding to miRNA is a reverse transcription primer for reverse transcription of the miRNA, an extension primer for extending the miRNA, an amplification primer set for amplifying the miRNA, or a nucleic acid probe for detecting the miRNA.


SEQUENCE LISTING (176 sequences)

SEQ ID NO: 1 - RNA, 22 nt, Homo sapiens
  source 1..22: mol_type = transcribed RNA; note = hsa-miR-3613-5p
tgttgtacttttttttttgttc

SEQ ID NO: 2 - RNA, 22 nt, Homo sapiens
  source 1..22: mol_type = transcribed RNA; note = hsa-miR-574-3p
cacgctcatgcacacacccaca

SEQ ID NO: 3 - RNA, 22 nt, Homo sapiens
  source 1..22: mol_type = transcribed RNA; note = hsa-miR-125b-5p
tccctgagaccctaacttgtga

SEQ ID NO: 4 - RNA, 22 nt, Homo sapiens
  source 1..22: mol_type = transcribed RNA; note = hsa-miR-223-5p
cgtgtatttgacaagctgagtt

SEQ ID NO: 5 - RNA, 24 nt, Homo sapiens
  source 1..24: mol_type = transcribed RNA; note = hsa-miR-3613-3p
acaaaaaaaaaagcccaacccttc

SEQ ID NO: 6 - RNA, 22 nt, Homo sapiens
  source 1..22: mol_type = transcribed RNA; note = hsa-miR-205-5p
tccttcattccaccggagtctg

SEQ ID NO: 7 - RNA, 23 nt, Homo sapiens
  source 1..23: mol_type = transcribed RNA; note = hsa-miR-941
cacccggctgtgtgcacatgtgc

SEQ ID NO: 8 - RNA, 23 nt, Homo sapiens
  source 1..23: mol_type = transcribed RNA; note = hsa-miR-324-3p
cccactgccccaggtgctgctgg

SEQ ID NO: 9 - RNA, 22 nt, Homo sapiens
  source 1..22: mol_type = transcribed RNA; note = hsa-miR-30e-5p
tgtaaacatccttgactggaag

SEQ ID NO: 10 - RNA, 22 nt, Homo sapiens
  source 1..22: mol_type = transcribed RNA; note = hsa-miR-193a-5p
tgggtctttgcgggcgagatga

SEQ ID NO: 11 - RNA, 21 nt, Homo sapiens
  source 1..21: mol_type = transcribed RNA; note = hsa-miR-106b-5p
taaagtgctgacagtgcagat

SEQ ID NO: 12 - RNA, 21 nt, Homo sapiens
  source 1..21: mol_type = transcribed RNA; note = hsa-miR-4433a-3p
acaggagtgggggtgggacat

SEQ ID NO: 13 - RNA, 22 nt, Homo sapiens
  source 1..22: mol_type = transcribed RNA; note = hsa-miR-29c-3p
tagcaccatttgaaatcggtta

SEQ ID NO: 14 - RNA, 22 nt, Homo sapiens
  source 1..22: mol_type = transcribed RNA; note = hsa-miR-190a-5p
tgatatgtttgatatattaggt

SEQ ID NO: 15 - RNA, 22 nt, Homo sapiens
  source 1..22: mol_type = transcribed RNA; note = hsa-miR-885-5p
tccattacactaccctgcctct

SEQ ID NO: 16 - RNA, 22 nt, Homo sapiens
  source 1..22: mol_type = transcribed RNA; note = hsa-miR-194-5p
tgtaacagcaactccatgtgga

SEQ ID NO: 17 - RNA, 22 nt, Homo sapiens
  source 1..22: mol_type = transcribed RNA; note = hsa-miR-29a-3p
tagcaccatctgaaatcggtta

SEQ ID NO: 18 - RNA, 21 nt, Homo sapiens
  source 1..21: mol_type = transcribed RNA; note = hsa-miR-142-5p
cataaagtagaaagcactact

SEQ ID NO: 19 - RNA, 22 nt, Homo sapiens
  source 1..22: mol_type = transcribed RNA; note = hsa-miR-483-5p
aagacgggaggaaagaagggag

SEQ ID NO: 20 - RNA, 23 nt, Homo sapiens
  source 1..23: mol_type = transcribed RNA; note = hsa-miR-142-3p
tgtagtgtttcctactttatgga

SEQ ID NO: 21 - RNA, 22 nt, Homo sapiens
  source 1..22: mol_type = transcribed RNA; note = hsa-miR-122-5p
tggagtgtgacaatggtgtttg

SEQ ID NO: 22 - RNA, 22 nt, Homo sapiens
  source 1..22: mol_type = transcribed RNA; note = hsa-miR-34a-5p
tggcagtgtcttagctggttgt

SEQ ID NO: 23 - RNA, 22 nt, Homo sapiens
  source 1..22: mol_type = transcribed RNA; note = hsa-miR-375-3p
tttgttcgttcggctcgcgtga

SEQ ID NO: 24 - RNA, 22 nt, Homo sapiens
  source 1..22: mol_type = transcribed RNA; note = hsa-miR-30d-5p
tgtaaacatccccgactggaag

SEQ ID NO: 25 - RNA, 22 nt, Homo sapiens
  source 1..22: mol_type = transcribed RNA; note = hsa-miR-451a
aaaccgttaccattactgagtt

SEQ ID NO: 26 - RNA, 22 nt, Homo sapiens
  source 1..22: mol_type = transcribed RNA; note = hsa-miR-148a-3p
tcagtgcactacagaactttgt

SEQ ID NO: 27 - RNA, 23 nt, Homo sapiens
  source 1..23: mol_type = transcribed RNA; note = hsa-miR-103a-3p
agcagcattgtacagggctatga

SEQ ID NO: 28 - RNA, 22 nt, Homo sapiens
  source 1..22: mol_type = transcribed RNA; note = hsa-miR-484
tcaggctcagtcccctcccgat

SEQ ID NO: 29 - RNA, 22 nt, Homo sapiens
  source 1..22: mol_type = transcribed RNA; note = hsa-miR-584-5p
ttatggtttgcctgggactgag

SEQ ID NO: 30 - RNA, 23 nt, Homo sapiens
  source 1..23: mol_type = transcribed RNA; note = hsa-miR-107
agcagcattgtacagggctatca

SEQ ID NO: 31 - RNA, 21 nt, Homo sapiens
  source 1..21: mol_type = transcribed RNA; note = hsa-miR-101-3p
tacagtactgtgataactgaa

SEQ ID NO: 32 - RNA, 21 nt, Homo sapiens
  source 1..21: mol_type = transcribed RNA; note = hsa-miR-143-3p
tgagatgaagcactgtagctc

SEQ ID NO: 33 - RNA, 22 nt, Homo sapiens
  source 1..22: mol_type = transcribed RNA; note = hsa-miR-382-5p
gaagttgttcgtggtggattcg

SEQ ID NO: 34 - RNA, 19 nt, Homo sapiens
  source 1..19: mol_type = transcribed RNA; note = hsa-miR-626
agctgtctgaaaatgtctt

SEQ ID NO: 35 - RNA, 23 nt, Homo sapiens
  source 1..23: mol_type = transcribed RNA; note = hsa-miR-432-5p
tcttggagtaggtcattgggtgg

SEQ ID NO: 36 - RNA, 21 nt, Homo sapiens
  source 1..21: mol_type = transcribed RNA; note = hsa-miR-4501
tatgtgacctcggatgaatca

SEQ ID NO: 37 - RNA, 22 nt, Homo sapiens
  source 1..22: mol_type = transcribed RNA; note = hsa-miR-22-3p
aagctgccagttgaagaactgt

SEQ ID NO: 38 - RNA, 21 nt, Homo sapiens
  source 1..21: mol_type = transcribed RNA; note = hsa-miR-27b-3p
ttcacagtggctaagttctgc

SEQ ID NO: 39 - RNA, 22 nt, Homo sapiens
  source 1..22: mol_type = transcribed RNA; note = hsa-miR-6785-5p
tgggagggcgtggatgatggtg

SEQ ID NO: 40 - RNA, 21 nt, Homo sapiens
  source 1..21: mol_type = transcribed RNA; note = hsa-miR-4433b-5p
atgtcccacccccactcctgt

SEQ ID NO: 41 - RNA, 21 nt, Homo sapiens
  source 1..21: mol_type = transcribed RNA; note = hsa-miR-7847-3p
cgtggaggacgaggaggaggc

SEQ ID NO: 42 - RNA, 21 nt, Homo sapiens
  source 1..21: mol_type = transcribed RNA; note = hsa-miR-128-3p
tcacagtgaaccggtctcttt

SEQ ID NO: 43 - RNA, 23 nt, Homo sapiens
  source 1..23: mol_type = transcribed RNA; note = hsa-miR-10b-5p
taccctgtagaaccgaatttgtg

SEQ ID NO: 44 - RNA, 22 nt, Homo sapiens
  source 1..22: mol_type = transcribed RNA; note = hsa-miR-5007-3p
atcatatgaaccaaactctaat

SEQ ID NO: 45 - RNA, 23 nt, Homo sapiens
  source 1..23: mol_type = transcribed RNA; note = hsa-miR-6728-5p
ttgggatggtaggaccagagggg

SEQ ID NO: 46 - RNA, 24 nt, Homo sapiens
  source 1..24: mol_type = transcribed RNA; note = hsa-miR-155-5p
ttaatgctaatcgtgataggggtt

SEQ ID NO: 47 - RNA, 22 nt, Homo sapiens
  source 1..22: mol_type = transcribed RNA; note = hsa-miR-99b-5p
cacccgtagaaccgaccttgcg

SEQ ID NO: 48 - RNA, 22 nt, Homo sapiens
  source 1..22: mol_type = transcribed RNA; note = hsa-miR-10b-3p
acagattcgattctaggggaat

SEQ ID NO: 49 - RNA, 22 nt, Homo sapiens
  source 1..22: mol_type = transcribed RNA; note = hsa-miR-2115-3p
catcagaattcatggaggctag

SEQ ID NO: 50 - RNA, 22 nt, Homo sapiens
  source 1..22: mol_type = transcribed RNA; note = hsa-miR-4782-5p
ttctggatatgaagacaatcaa

SEQ ID NO: 51 - RNA, 23 nt, Homo sapiens
  source 1..23: mol_type = transcribed RNA; note = hsa-miR-664a-3p
tattcatttatccccagcctaca

SEQ ID NO: 52 - RNA, 21 nt, Homo sapiens
  source 1..21: mol_type = transcribed RNA; note = hsa-miR-222-3p
agctacatctggctactgggt

SEQ ID NO: 53 - RNA, 21 nt, Homo sapiens
  source 1..21: mol_type = transcribed RNA; note = hsa-miR-5189-3p
tgccaaccgtcagagcccaga

SEQ ID NO: 54 - RNA, 22 nt, Homo sapiens
  source 1..22: mol_type = transcribed RNA; note = hsa-miR-6855-3p
agactgaccttcaaccccacag

SEQ ID NO: 55 - RNA, 23 nt, Homo sapiens
  source 1..23: mol_type = transcribed RNA; note = hsa-miR-20a-5p
taaagtgcttatagtgcaggtag

SEQ ID NO: 56 - RNA, 22 nt, Homo sapiens
  source 1..22: mol_type = transcribed RNA; note = hsa-miR-4798-5p
ttcggtatactttgtgaattgg

SEQ ID NO: 57 - RNA, 22 nt, Homo sapiens
  source 1..22: mol_type = transcribed RNA; note = hsa-miR-98-5p
tgaggtagtaagttgtattgtt

SEQ ID NO: 58 - RNA, 23 nt, Homo sapiens
  source 1..23: mol_type = transcribed RNA; note = hsa-miR-18a-5p
taaggtgcatctagtgcagatag

SEQ ID NO: 59 - RNA, 22 nt, Homo sapiens
  source 1..22: mol_type = transcribed RNA; note = hsa-miR-6749-5p
tcgggcctggggttgggggagc

SEQ ID NO: 60 - RNA, 23 nt, Homo sapiens
  source 1..23: mol_type = transcribed RNA; note = hsa-miR-4254
gcctggagctactccaccatctc

SEQ ID NO: 61 - RNA, 22 nt, Homo sapiens
  source 1..22: mol_type = transcribed RNA; note = hsa-miR-518a-3p
gaaagcgcttccctttgctgga

SEQ ID NO: 62 - RNA, 20 nt, Homo sapiens
  source 1..20: mol_type = transcribed RNA; note = hsa-miR-326
cctctgggcccttcctccag

SEQ ID NO: 63 - RNA, 22 nt, Homo sapiens
  source 1..22: mol_type = transcribed RNA; note = hsa-miR-4440
tgtcgtggggcttgctggcttg

SEQ ID NO: 64 - RNA, 23 nt, Homo sapiens
  source 1..23: mol_type = transcribed RNA; note = hsa-miR-19b-3p
tgtgcaaatccatgcaaaactga

SEQ ID NO: 65 - RNA, 23 nt, Homo sapiens
  source 1..23: mol_type = transcribed RNA; note = hsa-miR-885-3p
aacattcattgctgtcggtgggt

SEQ ID NO: 66 - RNA, 21 nt, Homo sapiens
  source 1..21: mol_type = transcribed RNA; note = hsa-miR-483-3p
tcactcctctcctcccgtctt

SEQ ID NO: 67 - RNA, 22 nt, Homo sapiens
  source 1..22: mol_type = transcribed RNA; note = hsa-miR-2276-3p
tctgcaagtgtcagaggcgagg

SEQ ID NO: 68 - RNA, 23 nt, Homo sapiens
  source 1..23: mol_type = transcribed RNA; note = hsa-miR-181b-5p
aacattcattgctgtcggtgggt

SEQ ID NO: 69 - RNA, 22 nt, Homo sapiens
  source 1..22: mol_type = transcribed RNA; note = hsa-miR-196a-5p
taggtagtttcatgttgttggg

SEQ ID NO: 70 - RNA, 21 nt, Homo sapiens
  source 1..21: mol_type = transcribed RNA; note = hsa-miR-4322
ctgtgggctcagcgcgtgggg

SEQ ID NO: 71 - RNA, 22 nt, Homo sapiens
  source 1..22: mol_type = transcribed RNA; note = hsa-miR-4780
acccttgagcctgatccctagc

SEQ ID NO: 72 - RNA, 23 nt, Homo sapiens
  source 1..23: mol_type = transcribed RNA; note = hsa-miR-668-3p
tgtcactcggctcggcccactac

SEQ ID NO: 73 - RNA, 22 nt, Homo sapiens
  source 1..22: mol_type = transcribed RNA; note = hsa-miR-196b-5p
taggtagtttcctgttgttggg

SEQ ID NO: 74 - RNA, 23 nt, Homo sapiens
  source 1..23: mol_type = transcribed RNA; note = hsa-miR-17-5p
caaagtgcttacagtgcaggtag

SEQ ID NO: 75 - RNA, 22 nt, Homo sapiens
  source 1..22: mol_type = transcribed RNA; note = hsa-miR-1306-5p
ccacctcccctgcaaacgtcca

SEQ ID NO: 76 - RNA, 22 nt, Homo sapiens
  source 1..22: mol_type = transcribed RNA; note = hsa-miR-653-3p
ttcactggagtttgtttcaata

SEQ ID NO: 77 - RNA, 22 nt, Homo sapiens
  source 1..22: mol_type = transcribed RNA; note = hsa-miR-4502
gctgatgatgatggtgctgaag

SEQ ID NO: 78 - RNA, 24 nt, Homo sapiens
  source 1..24: mol_type = transcribed RNA; note = hsa-miR-3609
caaagtgatgagtaatactggctg

SEQ ID NO: 79 - RNA, 22 nt, Homo sapiens
  source 1..22: mol_type = transcribed RNA; note = hsa-miR-3126-3p
catctggcatccgtcacacaga

SEQ ID NO: 80 - RNA, 23 nt, Homo sapiens
  source 1..23: mol_type = transcribed RNA; note = hsa-miR-514a-5p
tactctggagagtgacaatcatg

SEQ ID NO: 81 - RNA, 24 nt, Homo sapiens
  source 1..24: mol_type = transcribed RNA; note = hsa-miR-3662
gaaaatgatgagtagtgactgatg

SEQ ID NO: 82 - RNA, 23 nt, Homo sapiens
  source 1..23: mol_type = transcribed RNA; note = hsa-miR-8087
gaagacttcttggattacagggg

SEQ ID NO: 83 - RNA, 22 nt, Homo sapiens
  source 1..22: mol_type = transcribed RNA; note = hsa-miR-133a-3p
tttggtccccttcaaccagctg

SEQ ID NO: 84 - RNA, 21 nt, Homo sapiens
  source 1..21: mol_type = transcribed RNA; note = hsa-miR-4540
ttagtcctgcctgtaggttta

SEQ ID NO: 85 - RNA, 22 nt, Homo sapiens
  source 1..22: mol_type = transcribed RNA; note = hsa-miR-203a-3p
gtgaaatgtttaggaccactag

SEQ ID NO: 86 - RNA, 21 nt, Homo sapiens
  source 1..21: mol_type = transcribed RNA; note = hsa-miR-3666
cagtgcaagtgtagatgccga

SEQ ID NO: 87 - RNA, 21 nt, Homo sapiens
  source 1..21: mol_type = transcribed RNA; note = hsa-miR-4803
taacataatagtgtggattga

SEQ ID NO: 88 - RNA, 22 nt, Homo sapiens
  source 1..22: mol_type = transcribed RNA; note = hsa-miR-138-1-3p
gctacttcacaacaccagggcc

SEQ ID NO: 89 - RNA, 22 nt, Homo sapiens
  source 1..22: mol_type = transcribed RNA; note = hsa-miR-5700
taatgcattaaattattgaagg

SEQ ID NO: 90 - RNA, 22 nt, Homo sapiens
  source 1..22: mol_type = transcribed RNA; note = hsa-miR-22-5p
agttcttcagtggcaagcttta

SEQ ID NO: 91 - RNA, 22 nt, Homo sapiens
  source 1..22: mol_type = transcribed RNA; note = hsa-miR-5695
actccaagaagaatctagacag

SEQ ID NO: 92 - RNA, 22 nt, Homo sapiens
  source 1..22: mol_type = transcribed RNA; note = hsa-miR-1-3p
tggaatgtaaagaagtatgtat

SEQ ID NO: 93 - RNA, 23 nt, Homo sapiens
  source 1..23: mol_type = transcribed RNA; note = hsa-miR-365a-5p
agggacttttgggggcagatgtg

SEQ ID NO: 94 - RNA, 22 nt, Homo sapiens
  source 1..22: mol_type = transcribed RNA; note = hsa-miR-6811-5p
atgcaggcctgtgtacagcact

SEQ ID NO: 95 - RNA, 22 nt, Homo sapiens
  source 1..22: mol_type = transcribed RNA; note = hsa-miR-4661-5p
aactagctctgtggatcctgac

SEQ ID NO: 96 - RNA, 23 nt, Homo sapiens
  source 1..23: mol_type = transcribed RNA; note = hsa-miR-574-5p
tgagtgtgtgtgtgtgagtgtgt

SEQ ID NO: 97 - RNA, 22 nt, Homo sapiens
  source 1..22: mol_type = transcribed RNA; note = hsa-miR-505-5p
gggagccaggaagtattgatgt

SEQ ID NO: 98 - RNA, 21 nt, Homo sapiens
  source 1..21: mol_type = transcribed RNA; note = hsa-miR-4771
agcagacttgacctacaatta

SEQ ID NO: 99 - RNA, 22 nt, Homo sapiens
  source 1..22: mol_type = transcribed RNA; note = hsa-miR-3163
tataaaatgagggcagtaagac

SEQ ID NO: 100 - RNA, 21 nt, Homo sapiens
  source 1..21: mol_type = transcribed RNA; note = hsa-miR-31-5p
aggcaagatgctggcatagct

SEQ ID NO: 101 - RNA, 22 nt, Homo sapiens
  source 1..22: mol_type = transcribed RNA; note = hsa-miR-20a-3p
actgcattatgagcacttaaag

SEQ ID NO: 102 - RNA, 22 nt, Homo sapiens
  source 1..22: mol_type = transcribed RNA; note = hsa-miR-4777-3p
atacctcatctagaatgctgta

SEQ ID NO: 103 - RNA, 21 nt, Homo sapiens
  source 1..21: mol_type = transcribed RNA; note = hsa-miR-552-5p
gtttaaccttttgcctgttgg

SEQ ID NO: 104 - RNA, 22 nt, Homo sapiens
  source 1..22: mol_type = transcribed RNA; note = hsa-miR-3941
ttacacacaactgaggatcata

SEQ ID NO: 105 - RNA, 22 nt, Homo sapiens
  source 1..22: mol_type = transcribed RNA; note = hsa-miR-6781-3p
tgcctcttttccacggcctcag

SEQ ID NO: 106 - RNA, 23 nt, Homo sapiens
  source 1..23: mol_type = transcribed RNA; note = hsa-miR-550a-5p
agtgcctgagggagtaagagccc

SEQ ID NO: 107 - RNA, 22 nt, Homo sapiens
  source 1..22: mol_type = transcribed RNA; note = hsa-miR-16-5p
tagcagcacgtaaatattggcg

SEQ ID NO: 108 - RNA, 22 nt, Homo sapiens
  source 1..22: mol_type = transcribed RNA; note = hsa-let-7b-5p
tgaggtagtaggttgtgtggtt

SEQ ID NO: 109 - RNA, 23 nt, Homo sapiens
  source 1..23: mol_type = transcribed RNA; note = hsa-miR-423-5p
tgaggggcagagagcgagacttt

SEQ ID NO: 110 - RNA, 22 nt, Homo sapiens
  source 1..22: mol_type = transcribed RNA; note = hsa-let-7a-5p
tgaggtagtaggttgtatagtt

SEQ ID NO: 111 - RNA, 22 nt, Homo sapiens
  source 1..22: mol_type = transcribed RNA; note = hsa-let-7i-5p
tgaggtagtagtttgtgctgtt

SEQ ID NO: 112 - RNA, 21 nt, Homo sapiens
  source 1..21: mol_type = transcribed RNA; note = hsa-miR-486-3p
cggggcagctcagtacaggat

SEQ ID NO: 113 - RNA, 22 nt, Homo sapiens
  source 1..22: mol_type = transcribed RNA; note = hsa-let-7f-5p
tgaggtagtagattgtatagtt

SEQ ID NO: 114 - RNA, 22 nt, Homo sapiens
  source 1..22: mol_type = transcribed RNA; note = hsa-miR-126-3p
tcgtaccgtgagtaataatgcg

SEQ ID NO: 115 - RNA, 22 nt, Homo sapiens
  source 1..22: mol_type = transcribed RNA; note = hsa-miR-25-3p
cattgcacttgtctcggtctga

SEQ ID NO: 116 - RNA, 22 nt, Homo sapiens
  source 1..22: mol_type = transcribed RNA; note = hsa-miR-146a-5p
tgagaactgaattccatgggtt

SEQ ID NO: 117 - RNA, 22 nt, Homo sapiens
  source 1..22: mol_type = transcribed RNA; note = hsa-miR-21-5p
tagcttatcagactgatgttga

SEQ ID NO: 118 - RNA, 23 nt, Homo sapiens
  source 1..23: mol_type = transcribed RNA; note = hsa-miR-425-5p
aatgacacgatcactcccgttga

SEQ ID NO: 119 - RNA, 23 nt, Homo sapiens
  source 1..23: mol_type = transcribed RNA; note = hsa-miR-93-5p
caaagtgctgttcgtgcaggtag

SEQ ID NO: 120 - RNA, 22 nt, Homo sapiens
  source 1..22: mol_type = transcribed RNA; note = hsa-miR-150-5p
tctcccaacccttgtaccagtg

SEQ ID NO: 121 - RNA, 23 nt, Homo sapiens
  source 1..23: mol_type = transcribed RNA; note = hsa-miR-191-5p
caacggaatcccaaaagcagctg

SEQ ID NO: 122 - RNA, 24 nt, Homo sapiens
  source 1..24: mol_type = transcribed RNA; note = hsa-miR-125a-5p
tccctgagaccctttaacctgtga

SEQ ID NO: 123 - RNA, 23 nt, Homo sapiens
  source 1..23: mol_type = transcribed RNA; note = hsa-miR-342-3p
tctcacacagaaatcgcacccgt

SEQ ID NO: 124 - RNA, 23 nt, Homo sapiens
  source 1..23: mol_type = transcribed RNA; note = hsa-miR-221-3p
agctacattgtctgctgggtttc

SEQ ID NO: 125 - RNA, 21 nt, Homo sapiens
  source 1..21: mol_type = transcribed RNA; note = hsa-miR-26b-5p
ttcaagtaattcaggataggt

SEQ ID NO: 126 - RNA, 21 nt, Homo sapiens
  source 1..21: mol_type = transcribed RNA; note = hsa-miR-23a-3p
atcacattgccagggatttcc

SEQ ID NO: 127 - RNA, 22 nt, Homo sapiens
  source 1..22: mol_type = transcribed RNA; note = hsa-miR-197-3p
ttcaccaccttctccacccagc

SEQ ID NO: 128 - RNA, 22 nt, Homo sapiens
  source 1..22: mol_type = transcribed RNA; note = hsa-miR-24-3p
tggctcagttcagcaggaacag

SEQ ID NO: 129 - RNA, 22 nt, Homo sapiens
  source 1..22: mol_type = transcribed RNA; note = hsa-let-7d-3p
ctatacgacctgctgcctttct

SEQ ID NO: 130 - RNA, 22 nt, Homo sapiens
  source 1..22: mol_type = transcribed RNA; note = hsa-miR-328-3p
ctggccctctctgcccttccgt

SEQ ID NO: 131 - RNA, 22 nt, Homo sapiens
  source 1..22: mol_type = transcribed RNA; note = hsa-miR-320b
aaaagctgggttgagagggcaa

SEQ ID NO: 132 - RNA, 21 nt, Homo sapiens
  source 1..21: mol_type = transcribed RNA; note = hsa-miR-3130-5p
tacccagtctccggtgcagcc

SEQ ID NO: 133 - RNA, 22 nt, Homo sapiens
  source 1..22: mol_type = transcribed RNA; note = hsa-miR-744-5p
tgcggggctagggctaacagca

SEQ ID NO: 134 - RNA, 22 nt, Homo sapiens
  source 1..22: mol_type = transcribed RNA; note = hsa-miR-889-5p
aatggctgtccgtagtatggtc

SEQ ID NO: 135 - RNA, 22 nt, Homo sapiens
  source 1..22: mol_type = transcribed RNA; note = hsa-miR-625-3p
gactatagaactttccccctca

SEQ ID NO: 136 - RNA, 22 nt, Homo sapiens
  source 1..22: mol_type = transcribed RNA; note = hsa-miR-660-5p
tacccattgcatatcggagttg

SEQ ID NO: 137 - RNA, 22 nt, Homo sapiens
  source 1..22: mol_type = transcribed RNA; note = hsa-let-7e-5p
tgaggtaggaggttgtatagtt

SEQ ID NO: 138 - RNA, 23 nt, Homo sapiens
  source 1..23: mol_type = transcribed RNA; note = hsa-miR-181a-5p
aacattcaacgctgtcggtgagt

SEQ ID NO: 139 - RNA, 22 nt, Homo sapiens
  source 1..22: mol_type = transcribed RNA; note = hsa-miR-28-3p
cactagattgtgagctcctgga

SEQ ID NO: 140 - RNA, 23 nt, Homo sapiens
  source 1..23: mol_type = transcribed RNA; note = hsa-miR-4732-5p
tgtagagcagggagcaggaagct

SEQ ID NO: 141 - RNA, 23 nt, Homo sapiens
  source 1..23: mol_type = transcribed RNA; note = hsa-miR-139-3p
tggagacgcggccctgttggagt

SEQ ID NO: 142 - RNA, 22 nt, Homo sapiens
  source 1..22: mol_type = transcribed RNA; note = hsa-miR-324-5p
cgcatcccctagggcattggtg

SEQ ID NO: 143 - RNA, 22 nt, Homo sapiens
  source 1..22: mol_type = transcribed RNA; note = hsa-miR-127-3p
tcggatccgtctgagcttggct

SEQ ID NO: 144 - RNA, 22 nt, Homo sapiens
  source 1..22: mol_type = transcribed RNA; note = hsa-miR-16-2-3p
ccaatattactgtgctgcttta

SEQ ID NO: 145 - RNA, 22 nt, Homo sapiens
  source 1..22: mol_type = transcribed RNA; note = hsa-miR-186-5p
caaagaattctccttttgggct

SEQ ID NO: 146 - RNA, 25 nt, Homo sapiens
  source 1..25: mol_type = transcribed RNA; note = hsa-miR-6797-5p
aggagggaaggggctgagaacagga

SEQ ID NO: 147 - RNA, 21 nt, Homo sapiens
  source 1..21: mol_type = transcribed RNA; note = hsa-miR-27a-3p
ttcacagtggctaagttccgc

SEQ ID NO: 148 - RNA, 22 nt, Homo sapiens
  source 1..22: mol_type = transcribed RNA; note = hsa-miR-425-3p
atcgggaatgtcgtgtccgccc

SEQ ID NO: 149 - RNA, 22 nt, Homo sapiens
  source 1..22: mol_type = transcribed RNA; note = hsa-miR-532-5p; note = hsa-miR-532-5p
catgccttgagtgtaggaccgt

SEQ ID NO: 150 - RNA, 22 nt, Homo sapiens
  source 1..22: mol_type = transcribed RNA; note = hsa-miR-320a-3p
aaaagctgggttgagagggcga

SEQ ID NO: 151 - RNA, 17 nt, Homo sapiens
  source 1..17: mol_type = transcribed RNA; note = hsa-miR-3178
ggggcgcggccggatcg

SEQ ID NO: 152 - RNA, 22 nt, Homo sapiens
  source 1..22: mol_type = transcribed RNA; note = hsa-miR-361-5p
ttatcagaatctccaggggtac

SEQ ID NO: 153 - RNA, 23 nt, Homo sapiens
  source 1..23: mol_type = transcribed RNA; note = hsa-miR-375-5p
gcgacgagcccctcgcacaaacc

SEQ ID NO: 154 - RNA, 22 nt, Homo sapiens
  source 1..22: mol_type = transcribed RNA; note = hsa-miR-148a-5p
aaagttctgagacactccgact

SEQ ID NO: 155 - RNA, 23 nt, Homo sapiens
  source 1..23: mol_type = transcribed RNA; note = hsa-miR-6738-5p
cgaggggtagaagagcacagggg

SEQ ID NO: 156 - RNA, 22 nt, Homo sapiens
  source 1..22: mol_type = transcribed RNA; note = hsa-let-7g-5p
tgaggtagtagtttgtacagtt

SEQ ID NO: 157 - RNA, 22 nt, Homo sapiens
  source 1..22: mol_type = transcribed RNA; note = hsa-let-7b-3p
ctatacaacctactgccttccc

SEQ ID NO: 158 - RNA, 20 nt, Homo sapiens
  source 1..20: mol_type = transcribed RNA; note = hsa-miR-6794-3p
ctcactctcagtccctccct

SEQ ID NO: 159 - RNA, 18 nt, Homo sapiens
  source 1..18: mol_type = transcribed RNA; note = hsa-miR-4302
ccagtgtggctcagcgag

SEQ ID NO: 160 - RNA, 22 nt, Homo sapiens
  source 1..22: mol_type = transcribed RNA; note = hsa-miR-5586-5p
tatccagcttgttactatatgc

SEQ ID NO: 161 - RNA, 21 nt, Homo sapiens
  source 1..21: mol_type = transcribed RNA; note = hsa-miR-629-5p
tgggtttacgttgggagaact

SEQ ID NO: 162 - RNA, 22 nt, Homo sapiens
  source 1..22: mol_type = transcribed RNA; note = hsa-miR-378a-3p
actggacttggagtcagaaggc

SEQ ID NO: 163 - RNA, 21 nt, Homo sapiens
  source 1..21: mol_type = transcribed RNA; note = hsa-miR-606
aaactactgaaaatcaaagat

SEQ ID NO: 164 - RNA, 22 nt, Homo sapiens
  source 1..22: mol_type = transcribed RNA; note = hsa-miR-92b-3p
tattgcactcgtcccggcctcc

SEQ ID NO: 165 - RNA, 22 nt, Homo sapiens
  source 1..22: mol_type = transcribed RNA; note = hsa-miR-4778-5p
aattctgtaaaggaagaagagg

SEQ ID NO: 166 - RNA, 23 nt, Homo sapiens
  source 1..23: mol_type = transcribed RNA; note = hsa-miR-3617-3p
catcagcaccctatgtcctttct

SEQ ID NO: 167 - RNA, 24 nt, Homo sapiens
  source 1..24: mol_type = transcribed RNA; note = hsa-miR-8060
ccatgaagcagtgggtaggaggac

SEQ ID NO: 168 - RNA, 23 nt, Homo sapiens
  source 1..23: mol_type = transcribed RNA; note = hsa-miR-423-3p
agctcggtctgaggcccctcagt

SEQ ID NO: 169 - RNA, 20 nt, Homo sapiens
  source 1..20: mol_type = transcribed RNA; note = hsa-miR-6083
cttatatcagaggctgtggg

SEQ ID NO: 170 - RNA, 22 nt, Homo sapiens
  source 1..22: mol_type = transcribed RNA; note = hsa-miR-1298-5p
ttcattcggctgtccagatgta

SEQ ID NO: 171 - RNA, 23 nt, Homo sapiens
  source 1..23: mol_type = transcribed RNA; note = hsa-miR-23b-3p
atcacattgccagggattaccac

SEQ ID NO: 172 - RNA, 23 nt, Homo sapiens
  source 1..23: mol_type = transcribed RNA; note = hsa-miR-339-5p
tccctgtcctccaggagctcacg

SEQ ID NO: 173 - RNA, 22 nt, Homo sapiens
  source 1..22: mol_type = transcribed RNA; note = hsa-miR-1287-3p
ctctagccacagatgcagtgat

SEQ ID NO: 174 - RNA, 22 nt, Homo sapiens
  source 1..22: mol_type = transcribed RNA; note = hsa-miR-199a-3p
acagtagtctgcacattggtta

SEQ ID NO: 175 - RNA, 23 nt, Homo sapiens
  source 1..23: mol_type = transcribed RNA; note = hsa-miR-139-3p
tggagacgcggccctgttggagt

SEQ ID NO: 176 - RNA, 22 nt, Homo sapiens
  source 1..22: mol_type = transcribed RNA; note = hsa-miR-486-5p
tcctgtactgagctgccccgag